(19) United States
(12) Patent Application Publication
(10) Pub. No.: US 2008/0009416 A1
(43) Pub. Date: Jan. 10, 2008

(54) THIAZOLIUM COMPOUNDS AND USES THEREOF

(75) Inventors: Jeffrey L. Selph, Cary, NC (US); John J. Partridge, Chapel Hill, NC (US); John F. Reinhard, Billerica, MA (US)

Correspondence Address: MYERS BIGEL SIBLEY & SAJOVEC, PO BOX 37428, RALEIGH, NC, 27627, US

(73) Assignee: Mycosol, Inc.

(21) Appl. No.: 11/762,856

(22) Filed: Jun. 14, 2007

Related U.S. Application Data
(63) Continuation-in-part of application No. PCT/US05/45325, filed on Dec. 15, 2005.
(60) Provisional application No. 60/636,952, filed on Dec. 17, 2004.

(51) Int. Cl. A01N 25/28; A01N 43/78; A01P 3/00; A61P 37/00; C07D 277/20

(52) U.S. Cl. 504/156; 424/408; 424/59; 504/266; 514/365; 548/202

(57) ABSTRACT

Methods of controlling microbial growth utilizing thiazolium compounds are generally disclosed. Methods of controlling infestations relating to agricultural, industrial and marine uses through the use of thiazolium compounds are also disclosed. Further, methods of use of thiazolium compounds in medicine, particularly in the prophylaxis and treatment of inflammatory conditions, infectious conditions, as well as immune disorders are disclosed.

### CROSS REFERENCE TO RELATED APPLICATIONS

The present application is a continuation-in-part application and claims priority to PCT International Application Serial No. PCT/US2005/045325, filed Dec. 15, 2005, and published in English as PCT Publication No. WO 2006/065942 A2 on Jun. 22, 2006, which claims the benefit of U.S. Provisional Patent Application Ser. No. 60/636,952, filed Dec. 17, 2004, the entire contents of each of which are incorporated by reference herein.

### FIELD OF THE INVENTION

The present invention generally relates to methods of controlling microorganisms using thiazolium compounds. More specifically, the present invention relates to controlling microbial infestations relating to agricultural, industrial and marine uses. Additionally, the present invention relates to methods of using thiazolium compounds in medicine, particularly in the prophylaxis and treatment of inflammatory conditions, infectious conditions, as well as immune disorders.

### BACKGROUND OF THE INVENTION

Fungi includes organisms such as slime molds, mushrooms, smuts, rusts, mildews, molds, stinkhorns, puffballs, truffles and yeasts. Fungi are classified in their own kingdom because they absorb food in solution directly through their cell walls and reproduce through spores. Molds are a large group of fungi that are a common trigger for allergies and affect crops, plants and food. Molds can exist as tiny particles called “mold spores” present in indoor and outdoor air. There are more than 100,000 species in the world. Molds may grow anywhere they can find moisture sources. Common molds include Cladosporium, Penicillium, Aspergillus, Alternaria, Fusarium, Neurospora, Stachybotyrs and Mucor.

Soil-borne and seed-borne fungal pathogens of plants are responsible for severe economic losses in the agricultural and horticultural industries worldwide. These pathogens cause plant diseases such as seed decay, root/foot rot, seedling blight and wilt. Such diseases commonly reduce emergence, plant vigor and yield potential. Severe disease infection can kill emerging seedlings of an entire plant population, and result in a total loss of crop yield.

Solutions to the recurring problem of plant pathogens have been explored for decades. As particular crops become more abundant, and the area of land allocated for agriculture expands, there is an inherent need to employ more efficient and effective farming practices. As a result of increasing demand for crop production, farmers must often compromise their cultural practices by planting crops on sub-optimal land, or by increasing the frequency at which crops are planted in a specific location. In doing so, crop nutrients are depleted and specific crop pathogens, especially soil-borne or seed-borne pathogens, become more prevalent. Accordingly, it is increasingly difficult to sustain the health and productivity of a respective crop.

Various pathogens further cause diseases and illnesses in humans and animals presenting significant health risks.

The present invention relates to thiazolium derivatives, processes for their preparation, pharmaceutical formulations including the same, and their methods of use.

### SUMMARY OF THE PRESENT INVENTION

The present invention relates to methods and compositions comprising a thiazolium compound. One aspect of the present invention is a composition comprising formula I 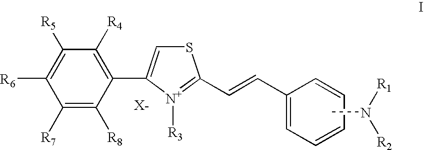
or a solvate thereof wherein said compound is substantially in the E, E configuration. The amino moieties may be in either the ortho, meta or para postions. X⁻ may be an anion, and thereby a thiazolium salt. The anion may be fluoride, chloride, bromide, iodide, halide, methanesulfonate (mesylate), benzenesulfonate (besylate), p-toluenesulfonate (tosylate), napthylate, m-nitrobenzenesulfonate (nosylate), para-aminobenzoate, lauryl sulfate, 2,4-dihydroxy benzophenone, or 2-(2-hydroxy-5′-methylphenyl)benzotriazole. R₁ and R₂, are independently selected from the group consisting of methyl, ethyl, C₁₋₁₀ alkyl (linear or branched), alkenes (linear or branched), or wherein R₁ and R₂ may be taken together with the nitrogen atom to which they are attached form pyrrolidino or piperidino rings; and R₃ is selected from the group consisting of methyl, ethyl, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties. R₃ may also be an organometallic compound such as organotin, organosilicon, or organogermanium. Additionally, R₃ may be (CH₂)ₙ-MR₉, wherein n is a number from 1 to 6, M is an organometallic compound such as tin, silicon, or germanium, and wherein R₉ is a selected from the group consisting of propyl, butyl, or any alkyl compound. In some embodiments, R₃ is selected from the group consisting of methyl, ethyl, C₁₋₁₀ alkyl (linear or branched), alkenes (linear or branched), alkynes, n-propyl, i-propyl, n-butyl, i-butyl, an organometallic compound, a polyalkylene glycol moiety, substituted and unsubstituted aryl moieties and substituted and unsubstituted benzyl moieties. R₄ through R₈ can be the same or different and can be selected from the group consisting of hydrogen, C₁₋₁₀ alkyl (linear or branched), representative examples of alkyl including, but not limited to, n-propyl, i-propyl, n-butyl, i-butyl, alkenes (linear or branched), alkynes, substituted and unsubstituted aryl moieties and substituted and unsubstituted benzyl moieties, hydroxy, alkoxy, SCH₃, (C₁-C₃) alkylthio, SH, (C₁-C₃) haloalkoxy, (C₁-C₃) perhaloalkoxy, NH₂, NH(lower alkyl), N(lower alkyl)₂, halogen, (C₁-C₃) haloalkyl, (C₁-C₃) perhaloalkyl, —CF₃, —CH₂CF₃, —CF₂CF₃, —CN, —NC, —OCN, —SCN, —NO, —NO₂, —N₃, —S(O) (lower alkyl), —S(O) (aryl), —S(O)₂ (lower alkyl), —S(O)₂ (aryl), S(O)₂ (alkoxyl), —S(O)₂(aryloxy), —S(O)NH₂; —S(O)₂NH-lower alkyl, —S(O)₂NH-aryl, —S(O)₂N-(lower alkyl)₂, —S(O)₂N-(aryl)₂, —C(O)Rₐ, —C(O)ORₐ, —C(O)NRₐR_{b}, —C(NH)NRₐR_{b}, —OC(O)Rₐ, —SC(O)Rₐ, —OC(O)ORₐ, —SC(O)ORₐ, —OC(O)NRₐR_{b}, —SC(O)NRₐR_{b}, —OC(NH)NRₐR_{b}, —SC(NH)NRₐR_{b}, —[NHC(O)]ₙRₐ, —[NHC(O)]ₙORₐ, —[NHC(O)]ₙNRₐR_{b} and —[NHC(NH)]ₙNRₐR_{b}, wherein n is an integer from 1 to 5, and wherein Rₐ and R_{b} can be the same or different and are independently selected from the group consisting of hydrogen, halogen, trifluoromethyl, alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamide, and any of R₄ through R₈ together can form a fused ring.

A further aspect of the present invention also relates to methods of controlling fungi and/or bacteria comprising administering a composition comprising any of the below formulas or a solvate thereof. 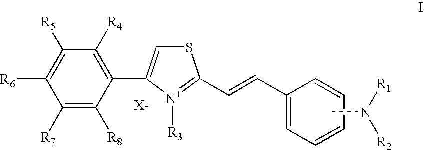
or a solvate thereof wherein said compound is substantially in the E, E configuration. The amino moieties may be in either the ortho, meta or para positions. The compound can also be in the E, Z or Z, Z configuration. X⁻ may be an anion, and thereby a thiazolium salt. The anion may be fluoride, chloride, bromide, iodide, halide, methanesulfonate (mesylate), benzenesulfonate (besylate), p-toluenesulfonate (tosylate), napthylate, m-nitrobenzenesulfonate (nosylate), para-aminobenzoate, lauryl sulfate, 2,4-dihydroxy benzophenone, or 2-(2-hydroxy-5′-methylphenyl)benzotriazole. R₁ and R₂, are independently selected from the group consisting of methyl, ethyl, C₁₋₁₀ alkyl (linear or branched), alkenes (linear or branched), or wherein R₁ and R₂ may be taken together with the nitrogen atom to which they are attached form pyrrolidino or piperidino rings; and R₃ is selected from the group consisting of methyl, ethyl, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties. R₃ may also be an organometallic compound such as organotin, organosilicon, or organogermanium. Additionally, R₃ may be (CH₂)ₙ-MR₉, wherein n is a number from 1 to 6, M is an organometallic compound such as tin, silicon, or germanium, and wherein R₉ is a selected from the group consisting of propyl, butyl, or any alkyl compound. In some embodiments, R₃ is selected from the group consisting of methyl, ethyl, C₁₋₁₀ alkyl (linear or branched), alkenes (linear or branched), alkynes, n-propyl, i-propyl, n-butyl, i-butyl, an organometallic compound, a polyalkylene glycol moiety, substituted and unsubstituted aryl moieties and substituted and unsubstituted benzyl moieties. R₄ through R₈ can be the same or different and can be selected from the group consisting of hydrogen, C₁₋₁₀ alkyl (linear or branched), representative examples of alkyl including, but not limited to, n-propyl, i-propyl, n-butyl, i-butyl, alkenes (linear or branched), alkynes, substituted and unsubstituted aryl moieties and substituted and unsubstituted benzyl moieties, hydroxy, alkoxy, SCH₃, (C₁-C₃) alkylthio, SH, (C₁-C₃) haloalkoxy, (C₁-C₃) perhaloalkoxy, NH₂, NH(lower alkyl), N(lower alkyl)₂, halogen, (C₁-C₃) haloalkyl, (C₁-C₃) perhaloalkyl, —CF₃, —CH₂CF₃, —CF₂CF₃, —CN, —NC, —OCN, —SCN, —NO, —NO₂, —N₃, —S(O) (lower alkyl), —S(O) (aryl), —S(O)₂ (lower alkyl), —S(O)₂ (aryl), S(O)₂ (alkoxyl), —S(O)₂(aryloxy), —S(O)NH₂; —S(O)₂NH-lower alkyl, —S(O)₂NH-aryl, —S(O)₂N-(lower alkyl)₂, —S(O)₂N-(aryl)₂, —C(O)Rₐ, —C(O)ORₐ, —C(O)NRₐR_{b}, —C(NH)NRₐR_{b}, —OC(O)Rₐ, —SC(O)Rₐ, —OC(O)ORₐ, —SC(O)ORₐ, —OC(O)NRₐR_{b}, —SC(O)NRₐR_{b}, —OC(NH)NRₐR_{b}, —SC(NH)NRₐR_{b}, —[NHC(O)]ₙRₐ, —[NHC(O)]ₙORₐ, —[NHC(O)]ₙNRₐR_{b} and —[NHC(NH)]ₙNRₐR_{b}, wherein n is an integer from 1 to 5, and wherein Rₐ and R_{b} can be the same or different and are independently selected from the group consisting of hydrogen, halogen, trifluoromethyl, alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamide, and any of R₄ through R₈ together can form a fused ring.

Aspects of the present invention also relate to articles of manufacture, substrates and/or materials including the compounds described herein. Articles of manufacture, substrates and/or materials include, but are not limited to, wood, air ducts, lumber, floorings, decks, buoys, seawalls, retaining walls, docks, pilings, watercrafts, boats, pipes, stucco, tiles, paint, insulation, roofs, roofing materials, building materials, metal, concrete and cement-based materials, plasters, asphalts, ceramics, stucco, sheetrock, grout, caulking, mortar, plastics, foam, glass, carpets, wallpaper, cloth, computer parts, food packaging, paper products, medical devices, petroleum processing, oil and natural gas extraction, metal working fluids, fasteners, adhesives, sealants, recreational water bodies, such as swimming pools, saunas, hot tubs, whirlpools, jacuzzis and spas, etc., and surfaces thereof, wall coverings, siding materials, flooring, filtration systems, cooling towers, personal care and/or hygiene products, cosmetics and other suitable articles of manufacture, substrates and/or materials.

Additional aspects of the present invention relate to methods of controlling algal, fungal, bacterial, viral, and/or parasitic growth and/or infections including applying compositions including the compounds of the present invention and a cosmetically, agriculturally or industrially acceptable carrier, excipient or diluent, in an amount effective to control the algal, fungal, bacterial, viral, and/or parasitic growth and/or infection.

Aspects of the present invention further relate to use of the compounds of the present invention for the preparation of a medicament for the treatment of an algal, fungal, bacterial, viral, and/or parasitic infection.

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. Unless otherwise defined, all terms, including technical and scientific terms used in the description, have the same meaning as commonly understood by one of ordinary skill in the art to which this invention belongs.

All publications, patent applications, patents and other references cited herein are incorporated by reference in their entireties for the teachings relevant to the sentence and/or paragraph in which the reference is presented.

As used herein, the term “microbe” or “microbial” refers to microscopic organisms that can exist as a single cell or cell clusters.

As used herein, the term “eliminating” refers to complete cessation of the specified activity.

As used herein, the term “reducing” or “reduce” refers to a decrease or diminishment in the specified activity of at least about 10%, 25%, 35%, 40%, 50%, 60%, 75%, 80%, 90%, 95% or more. In some embodiments, the reduction results in little or essentially no detectible activity (at most, an insignificant amount, e.g., less than about 10% or even 5%).

As used herein, the term “retarding the growth” or “retardation of growth” refers to reducing, delaying and/or hindering activity contributing to the growth of the microorganism.

As used herein, the terms “controlling the growth” refer to eliminating or retarding growth of the microorganism. Accordingly, compounds of the present invention possess biostatic and biocidal properties.

As used herein, the term “effective amount” refers to an amount of a compound or composition that is sufficient to produce the desired effect, which can be a therapeutic or agricultural effect. The effective amount will vary with the application for which the compound or composition is being employed, the microorganism and/or the age and physical condition of the subject, the severity of the condition, the duration of the treatment, the nature of any concurrent treatment, the pharmaceutically or agriculturally acceptable carrier used, and like factors within the knowledge and expertise of those skilled in the art. An appropriate “effective amount” in any individual case can be determined by one of ordinary skill in the art by reference to the pertinent texts and literature and/or by using routine experimentation. (See, for example for pharmaceutical applications, Remington, The Science And Practice of Pharmacy (20th Ed. 2000).

As used herein, the term “treat” refers to an action resulting in a reduction in the severity of the subject's condition or at least the condition is partially improved or ameliorated and/or that some alleviation, mitigation or decrease in at least one clinical symptom (or agricultural index for plants or comparable measure for industrial products) is achieved and/or there is a delay in the progression of the condition and/or prevention or delay of the onset of the condition. Thus, the term “treat” refers to both prophylactic and therapeutic treatment regimes.

“Alkyl” as used herein alone or as part of another group, refers to a straight or branched chain hydrocarbon including from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like.

“Lower alkyl” as used herein, is a subset of alkyl, in some embodiments preferred, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. Alkyl and loweralkyl groups may be unsubstituted or substituted one or more times with halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1 or 2.

“Alkoxy,” as used herein alone or as part of another group, refers to an alkyl group, as defined herein, appended to the parent molecular moiety through an oxy group. Representative examples of alkoxy include, but are not limited to, methoxy, ethoxy, propoxy, 2-propoxy, butoxy, tert-butoxy, pentyloxy, hexyloxy and the like.

“Acyl” or “Alkanoyl” as used herein alone or as p art of another group, refers to a —C(O)R radical, where R is any suitable substituent such as alkyl, alkenyl, alkynyl, aryl, alkylaryl, etc. as given herein.

As used herein, the term “controlled release” is intended to mean the release of a bio-active at a pre-selected or desired rate. This rate will vary depending upon the application. Desirable rates include fast or immediate release profiles as well as delayed, sustained or sequential release profiles. Combinations of release patterns, such as initial spiked release followed by lower levels of sustained release of the bio-active are also contemplated by the present invention.

As used herein, the term “bio-active” includes therapeutic agents such as pharmaceutical or pharmacological active agents, e.g., drugs and medicaments, as well as prophylactic agents, diagnostic agents and other chemicals or materials useful in treating or preventing conditions, infections and/or diseases. The compositions of the present invention are particularly effective in plants and other organisms.

As used herein, “photosensitive material” refers to all compositions and materials designed to block and/or absorb ultraviolet light. This term also refers to all photoprotective and photoresistant agents.

As herein used, “surfactant” refers to all compositions including surfactant salt compositions that are capable of forming emulsions, micro-emulsions, suspensions, etc.

The present invention relates to thiazolium derivatives, processes for their preparation, methods of their use and compositions comprising such derivatives.

One of the embodiments of the present invention includes a compound comprising: 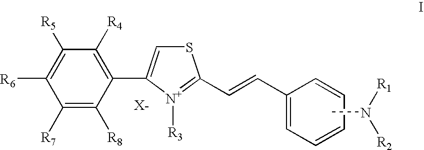
or a solvate thereof wherein said compound is substantially in the E, E configuration. The amino moieties may be in either the ortho, meta or para positions. The compound can also be in the E, Z or Z, Z configuration. X⁻ may be an anion, and thereby a thiazolium salt. The anion may be fluoride, chloride, bromide, iodide, halide, methanesulfonate (mesylate), benzenesulfonate (besylate), p-toluenesulfonate (tosylate), napthylate, m-nitrobenzenesulfonate (nosylate), para-aminobenzoate, lauryl sulfate, 2,4-dihydroxy benzophenone, or 2-(2-hydroxy-5′-methylphenyl)benzotriazole. R₁ and R₂, are independently selected from the group consisting of methyl, ethyl, C₁₋₁₀ alkyl (linear or branched), alkenes (linear or branched), or wherein R₁ and R₂ may be taken together with the nitrogen atom to which they are attached form pyrrolidino or piperidino rings; and R₃ is selected from the group consisting of methyl, ethyl, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties. R₃ may also be an organometallic compound such as organotin, organosilicon, or organogermanium. Additionally, R₃ may be (CH₂)ₙ-MR₉, wherein n is a number from 1 to 6, M is an organometallic compound such as tin, silicon, or germanium, and wherein R₉ is a selected from the group consisting of propyl, butyl, or any alkyl compound. In some embodiments, R₃ is selected from the group consisting of methyl, ethyl, C₁₋₁₀ alkyl (linear or branched), alkenes (linear or branched), alkynes, n-propyl, i-propyl, n-butyl, i-butyl, an organometallic compound, a polyalkylene glycol moiety, substituted and unsubstituted aryl moieties and substituted and unsubstituted benzyl moieties. R₄ through R₈ can be the same or different and can be selected from the group consisting of hydrogen, C₁₋₁₀ alkyl (linear or branched), representative examples of alkyl including, but not limited to, n-propyl, i-propyl, n-butyl, i-butyl, alkenes (linear or branched), alkynes, substituted and unsubstituted aryl moieties and substituted and unsubstituted benzyl moieties, hydroxy, alkoxy, SCH₃, (C₁-C₃) alkylthio, SH, (C₁-C₃) haloalkoxy, (C₁-C₃) perhaloalkoxy, NH₂, NH(lower alkyl), N(lower alkyl)₂, halogen, (C₁-C₃) haloalkyl, (C₁-C₃) perhaloalkyl, —CF₃, —CH₂CF₃, —CF₂CF₃, —CN, —NC, —OCN, —SCN, —NO, —NO₂, —N₃, —S(O) (lower alkyl), —S(O) (aryl), —S(O)₂ (lower alkyl), —S(O)₂ (aryl), S(O)₂ (alkoxyl), —S(O)₂(aryloxy), —S(O)NH₂; —S(O)₂NH-lower alkyl, —S(O)₂NH-aryl, —S(O)₂N-(lower alkyl)₂, —S(O)₂N-(aryl)₂, —C(O)Rₐ, —C(O)ORₐ, —C(O)NRₐR_{b}, —C(NH)NRₐR_{b}, —OC(O)Rₐ, —SC(O)Rₐ, —OC(O)ORₐ, —SC(O)ORₐ, —OC(O)NRₐR_{b}, —SC(O)NRₐR_{b}, —OC(NH)NRₐR_{b}, —SC(NH)NRₐR_{b}, —[NHC(O)]ₙRₐ, —[NHC(O)]ₙORₐ, —[NHC(O)]ₙNRₐR_{b} and —[NHC(NH)]ₙNRₐR_{b}, wherein n is an integer from 1 to 5, and wherein Rₐ and R_{b} can be the same or different and are independently selected from the group consisting of hydrogen, halogen, trifluoromethyl, alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamide, and any of R₄ through R₅ together can form a fused ring.

The compounds of the present invention are capable of existing as geometric isomers. All such isomers, individually and as mixtures, are included within the scope of the present invention for their agricultural, medical, industrial and marine uses.

The present invention also includes methods comprising administering a composition comprising any of the below formulas or a solvate thereof.

These compounds include the following: 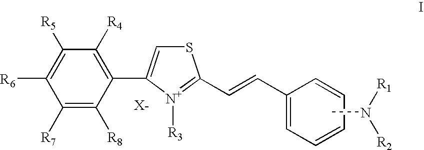
or a solvate thereof wherein said compound is substantially in the E, E configuration. The amino moieties may be in either the ortho, meta or para postions. The compound can also be in the E, Z or Z, Z configuration. X⁻ may be an anion, and thereby a thiazolium salt. The anion may be fluoride, chloride, bromide, iodide, halide, methanesulfonate (mesylate), benzenesulfonate (besylate), p-toluenesulfonate (tosylate), napthylate, m-nitrobenzenesulfonate (nosylate), para-aminobenzoate, lauryl sulfate, 2,4-dihydroxy benzophenone, or 2-(2-hydroxy-5′-methylphenyl)benzotriazole. R₁ and R₂, are independently selected from the group consisting of methyl, ethyl, C₁₋₁₀ alkyl (linear or branched), alkenes (linear or branched), or wherein R₁ and R₂ may be taken together with the nitrogen atom to which they are attached form pyrrolidino or piperidino rings; and R₃ is selected from the group consisting of methyl, ethyl, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties. R₃ may also be an organometallic compound such as organotin, organosilicon, or organogermanium. Additionally, R₃ may be (CH₂)ₙ-MR₉, wherein n is a number from 1 to 6, M is an organometallic compound such as tin, silicon, or germanium, and wherein R₉ is a selected from the group consisting of propyl, butyl, or any alkyl compound. In some embodiments, R₃ is selected from the group consisting of methyl, ethyl, C₁₋₁₀ alkyl (linear or branched), alkenes (linear or branched), alkynes, n-propyl, i-propyl, n-butyl, i-butyl, an organometallic compound, a polyalkylene glycol moiety, substituted and unsubstituted aryl moieties and substituted and unsubstituted benzyl moieties. R₄ through R₉ can be the same or different and can be selected from the group consisting of hydrogen, C₁₋₁₀ alkyl (linear or branched), representative examples of alkyl including, but not limited to, n-propyl, i-propyl, n-butyl, i-butyl, alkenes (linear or branched), alkynes, substituted and unsubstituted aryl moieties and substituted and unsubstituted benzyl moieties, hydroxy, alkoxy, SCH₃, (C₁-C₃) alkylthio, SH, (C₁-C₃) haloalkoxy, (C₁-C₃) perhaloalkoxy, NH₂, NH(lower alkyl), N(lower alkyl)₂, halogen, (C₁-C₃) haloalkyl, (C₁-C₃) perhaloalkyl, —CF₃, —CH₂CF₃, —CF₂CF₃, —CN, —NC, —OCN, —SCN, —NO, —NO₂, —N₃, —S(O) (lower alkyl), —S(O) (aryl), —S(O)₂ (lower alkyl), —S(O)₂ (aryl), S(O)₂ (alkoxyl), —S(O)₂(aryloxy), —S(O)NH₂; —S(O)₂NH-lower alkyl, —S(O)₂NH-aryl, —S(O)₂N-(lower alkyl)₂, —S(O)₂N-(aryl)₂, —C(O)Rₐ, —C(O)ORₐ, —C(O)NRₐR_{b}, —C(NH)NRₐR_{b}, —OC(O)Rₐ, —SC(O)Rₐ, —OC(O)ORₐ, —SC(O)ORₐ, —OC(O)NRₐR_{b}, —SC(O)NRₐR_{b}, —OC(NH)NRₐR_{b}, —SC(NH)NRₐR_{b}, —[NHC(O)]ₙRₐ, —[NHC(O)]ₙORₐ, —[NHC(O)]ₙNRₐR_{b} and —[NHC(NH)]ₙNRₐR_{b}, wherein n is an integer from 1 to 5, and wherein Rₐ and R_{b} can be the same or different and are independently selected from the group consisting of hydrogen, halogen, trifluoromethyl, alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamide, and any of R₄ through R₈ together can form a fused ring.

A synthetic scheme is outlined below and utilizes the procedures of Phillips and Burrows discussed in the U.S. Pat. Nos. 3,641,012; 3,851,060 and 3,883,658. Typically, commercially available substituted or unsubstituted phenyl methyl ketones (1) can be reacted with bromine in a non-polar solvent to produce the corresponding phenacyl bromides (2). Reaction of the reactive alpha-bromo ketones (2) with commercially available thioacetamide (3) in an protic solvent such as methanol with heat will afford the 2-methyl-4-phenylthiazoles (4). N-Alkylation of thiazoles (4) with alkyl halides such as methyl iodide (5) in aprotic solvents such as dimethylformamide and heat form the corresponding methiodide products (6), which are also named either N-alkyl-2-methyl-4-phenylthiazolium halides (6) or 3-alkyl-2-methyl-4-phenylthiazolium halides. Reaction of these thiazolium halides (6) with (N,N′-disubstituted)amino benzaldehydes (7) in a protic solvent such as methanol with a basic catalyst such as piperidine and heat then produces the desired 2-(N,N′-dialkylaminostyryl)-3-methyl-4-phenylthiazolium halides (8). To prepare a substantially pure E-isomer of the thiazolium salts, after reaction of the thiazolium halides with the benzaldehydes in a protic solvent such as methanol with a basic catalyst such as piperidine and heat, following with crystallization or chromatographic purification produces the desired E-2-(N,N′-dialkylaminostyryl)-3-methyl-4-phenylthiazolium halides.

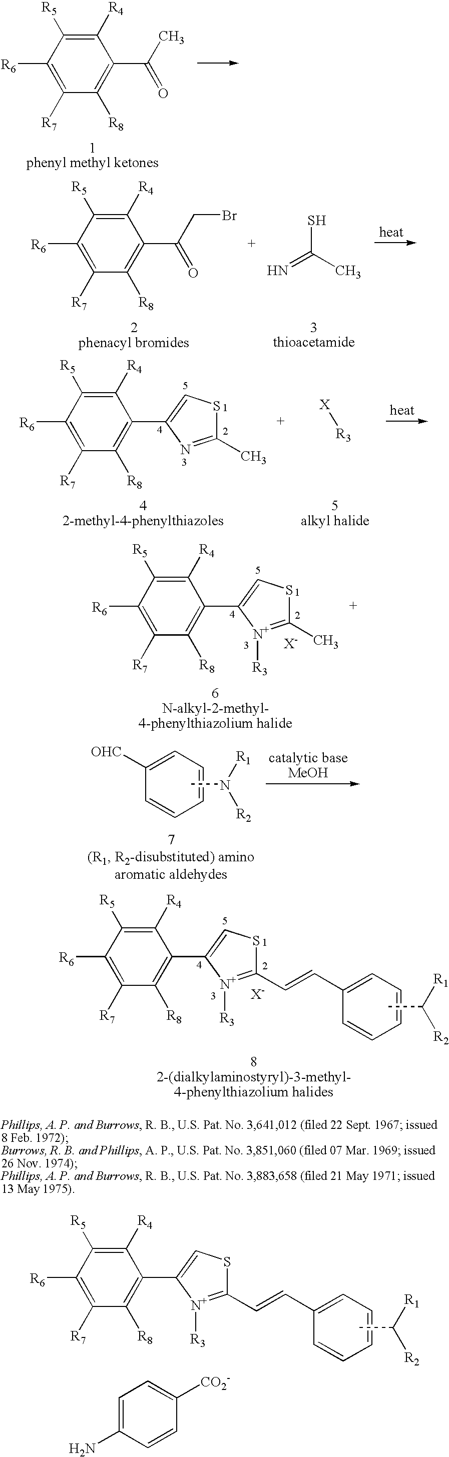

Another embodiment of the present invention can include the thiazolium compounds being encapsulated. As used herein the term “microcapsules” is intended to contemplate single molecules, encapsulated discrete particulate, multiparticulate, liquid multicore and homogeneously dissolved active components. The encapsulation method may provide either a water soluble or oil soluble active component encapsulated in a shell matrix of either a water or oil soluble material. The microencapsulated active component may be protected from oxidation and hydration, and may be released by melting, rupturing, biodegrading, or dissolving the surrounded shell matrix or by slow diffusion of the active component through the matrix. Microcapsules usually fall in the size range of between about 1 and 2000 microns, although smaller and larger sizes are known in the art.

The compound of the present invention may be placed in a microcapsule or hollow fiber type used for distribution. They may also be dispersed in a polymeric material or held as a liquid.

An active ingredient may be placed with the compound of the present invention in a microcapsule. Examples of the active ingredient having repellent activity may include triethylene glycol monohexyl ether and N,N-diethyl-m-triamide (or N,N diethyl-m-toluamide, DEET). Examples of the active ingredient having aromatic activity include geraniol, limonene, benzyl alcohol, esters of a C₆₋₂₀ hydrocarbon, ethers, aldehydes and alcoholic compounds. Examples of the active ingredient having pesticidal activity include insecticides such as salithion, diazinon and chlorpyrifos and bactericides such as thiophanate-methyl and captan.

Such constituents can be encapsulated, as is desired in the case of phase change materials. Such encapsulated constituents can further be encapsulated in microcapsules. The microcapsules can be made from a wide variety of materials, including polyethylene, polypropylenes, polyesters, polyvinyl chloride, polystyrene, tristarch acetates, polyethylene oxides, polypropylene oxides, polyvinylidene chloride or fluoride, polyvinyl alcohols, polyvinyl acetates, urethanes, polycarbonates, and polylactones. Further details on microencapsulation can be found in U.S. Pat. Nos. 5,589,194 and 5,433,953, the contents of which are incorporated herein in their entirety. Microcapsules suitable for use in the base materials of the present invention have diameters from about 1.0 to 2,000 microns.

No particular limitation is imposed on the shape for holding the active ingredient. In other words, there are various forms for holding the active ingredient by a holding mixture. Specific examples include microcapsules in which the surface of the active ingredient has been covered with the holding mixture; and products processed into a desired shape, each being obtained by kneading the active ingredient in the holding mixture or forming a uniform solution of the holding mixture and the active ingredient, dispersing the active ingredient in the holding mixture by the removal of the solvent or the like and then processing the dispersion into a desired shape such as single molecule, liquid, sphere, sheet, film, rod, pipe, thread, tape or chip. In addition, these processed products having a surface covered with a barrier layer for controlling the release of the active ingredient and those coated with an adhesive for improving applicability can be given as examples. As further examples, those obtained by filling the active ingredient in the holding mixture processed into a form of a capillary tube, heat sealing both ends of the capillary tube and then encapsulating the active ingredient therein; and those obtained by centrally cutting the above-mentioned capillary tube into two pieces, thereby having each one end as an opening.

The container formed of a holding mixture which container has an active ingredient enclosed therein as a liquid phase to secure uniform release ability over a long period of time. As such shape, tube-, bottle- or bag-shaped container is used generally.

When the mixture is formed into a container, the sustained release layer desirably has a thickness of at least about 0.002 mm for effecting stable sustained release. No particular problem may occur when the sustained release layer has a thickness not smaller than about 0.002 mm, but that ranging from about 0.005 mm to 5 mm can be used. When the thickness exceeds about 5 mm, the release amount of the compound tends to become too small.

For solids, the release surface area of the sustained release preparation formed of such a container is desirably about 0.001 cm² or larger. A range of from about 0.01 μm² to 1 cm² may be used.

When the active ingredient is enclosed and held in a container of the sustained release preparation, said container having been formed of a holding mixture, it may be enclosed in portions. The enclosed amount can be about 0.5 mg to 5 mg, and may be about 1 mg, 2 mg, 3 mg, or 4 mg.

As the shape of the container formed of a holding mixture, a tube, bottle and bag can be used. In the case of the tube-shaped preparation, that having an internal diameter of about 0.4 mm to 10 mm can be used. Internal diameters smaller than about 0.4 mm make it difficult to fill the active ingredient in the container, while those larger than about 10 mm make it difficult to conduct encapsulation. The bottle-shaped preparation is formed by blow molding or injection molding and generally has an internal volume of about 0.1 to 200 ml. The bottle having an internal volume less than about 0.1 ml cannot be formed easily, while that having an internal volume greater than about 200 ml is not economical because there is a large difference between the amount of the active ingredient filled therein and the internal volume. In the case of a bag-shaped preparation, the amount of the active ingredient filled in the bag is desirably about 1 mg to 100 g.

The biodegradable sustained-release preparation according to the first group of the present invention should retain its essential performance during application so that a pigment or dye, or various stabilizers such as ultraviolet absorber/blocker or antioxidant may be added to the holding mixture in order to improve the weather resistance. Alternatively, it is possible to add such an additive to the active ingredient enclosed in the container formed of a holding mixture.

In accordance with the present invention there is provided a microcapsule biocide such as a bacteriocide and/or fungicide composition comprising microcapsules each having a polyurea shell including as an integral part of said shell a photostable ultraviolet light absorbent compound or blocker compound having a log molar extinction coefficient of from about 2 to 5 with respect to radiation having wave lengths in the range of from about 270 to 350 nanometers and a liquid fill capable of slowly permeating the shell and comprising a thiazolium salt and a biological synergist therefor.

The entire microcapsule composition can include of about 60 to 90 percent of liquid fill and about 40 to 10 percent of shell wall, the liquid fill comprising about 5 to 40 percent of thiazolium salt, about 25 to 50 percent of biological synergist and about 20 to 40 percent of a water-immiscible organic solvent and the shell including as an integral part thereof about 0.5 to 20 percent of photostable ultraviolet light absorbent compound (all percentages being based on the weight of the entire microcapsule composition).

The thiazolium salt can remain inside the microcapsules while the composition is packaged and in storage, i.e., in a closed container due to the partial pressure of the thiazolium salt surrounding the microcapsules. When the product is applied as a biocide such as a bacteriocide and/or fungicide, the thiazolium salt, releases slowly (the actual speed of release depending upon the thickness and porosity of the capsule walls). The thiazolium salt is chemically stable during storage and after application until it permeates the capsule walls. At that time it becomes available as a bacteriocide and/or fungicide until degraded. Since the fill permeates the shell wall slowly, the microcapsule product has a long effective bacteriocide and/or fungicide life and may be stored for extended periods (e.g. for 6 months and more).

Suitable fill stabilizers absorb ultraviolet radiation in the range of about 270 to 350 nanometers and convert it to a harmless form. They have a high absorption coefficient in the near ultraviolet portion of the spectrum (e.g. a log molar extinction coefficient of from about 2 to 5) but only minimal absorption in the visible portion of the spectrum. They do not exhibit any substantial chemical reaction with the isocyanate groups and primary amine groups of the shell forming compounds during the microencapsulation process. Among the compounds which can be used as fill stabilizers are substituted benzophenones such as 2,4-dihydroxy benzophenone, 2-hydroxy-4-methoxy benzophenone, 2-hydroxy-4-octyloxy benzophenone, etc.; the benzotriazoles such as 2-(2-hydroxy-5′-methylphenyl)benzotriazole, 2-(3′,5′-diallyl-2′-hydroxylphenyl)benzotriazole, etc.; substituted acrylates such as ethyl 2-cyano-3,3-diphenyl acrylate, 2-ethylhexyl-2-cyano-3,3-diphenyl acetate, etc.; salicylates such as phenyl salicylates, 5-butyl phenyl salicylate, etc.; and nickel organic compounds such as nickel bis(octylphenol) sulfide, etc. Additional examples of each of these classes of fill stabilizers may be found in Kirk-Othmer, Encyclopedia of Chemical Technology. The fill stabilizers may comprise up to 5 percent, and are generally from about 0.01 to 2 percent, by weight of the microcapsule composition.

Embodiments of the invention also provide a process for controlling microbial activity by contacting the microorganism with an effective level of the compositions comprising thiazolium compound as recited throughout. Contact may be accomplished directly, for example, by atomization of the composition into the air in the form of a spray. Alternatively, compositions of the present invention may be provided in various other forms, for example in sheet materials carrying the microcapsules, (e.g. tapes coated or impregnated with the microcapsules) that may be placed in areas where the microbes may grow.

Another embodiment of the present invention may include heat sensitive materials that are excellent in preservation stability especially in resistance to light, and microcapsules having an ultraviolet absorber enclosed therein, which are applicable to various fields. Desirable constituents, which may be present in a base material, include materials that can absorb heat and protect an underlying material from overheating. Thermal energy is absorbed by the phase change of such materials without causing an increase in the temperature of these materials. Suitable phase change materials include paraffinic hydrocarbons, that is, straight chain hydrocarbons represented by the formula CnHn+₂, where n can range from 13 to 28. Other compounds which are suitable for phase change materials are 2,2-dimethyl-1,3-propane diol (DMP), 2-hydroxymethyl-2-methyl-1,3-propane diol (HMP) and similar compounds. Also useful are the fatty esters such as methyl palmitate. Phase change materials that can be used include paraffinic hydrocarbons.

Heat sensitive recording materials are well known which utilize a color forming reaction between a colorless or light-colored basic dye and an organic or inorganic color acceptor to obtain record images by thermally bringing the two chromogenic substances into contact with each other. Such heat sensitive recording materials are relatively inexpensive, are adapted for use with recording devices which are compact and easy to maintain, and have therefore found wide applications as recording media for facsimile systems, various computers, etc. In order to improve light resistance of heat sensitive recording materials a finely divided ultraviolet absorber or blocker can be added to the heat sensitive recording layer or protective layer.

Another embodiment of the present invention is to provide microcapsules which have excellent retainability of ultraviolet absorber, difficult to be ruptured at a usual pressure and are excellent in ultraviolet ray absorbing efficiency.

Embodiments of the present invention can include a heat sensitive recording material comprising a substrate, a recording layer formed over the substrate and containing a colorless or light-colored basic dye and a color acceptor, and a protective layer formed over the recording layer, the recording material being characterized in that microcapsules having an ultraviolet absorber enclosed therein and having substantially no color forming ability are incorporated in the protective layer.

Further, the present invention provides microcapsules having an ultraviolet absorber and as required an organic solvent enclosed therein, which have capsule wall film of synthetic resin and mean particle size of about 0.1 to 3 μm.

Embodiments of the present invention further provide attachment of polyalkylene moieties to the compounds described herein, which technique can be employed to reduce immunogenicity and/or extend the half-life of the native compounds discussed herein. Any conventional PEGylation method can be employed, provided that the PEGylated agent retains pharmaceutical activity. See also Schacht, E. H. et al. Poly(ethylene glycol) Chemistry and Biological Applications, American Chemical Society, San Francisco, Calif. 297-315 (1997).

Polyalkylene glycol is a biocompatible polymer where, as used herein, polyalkylene glycol refers to straight or branched polyalkylene glycol polymers such as polyethylene glycol, polypropylene glycol, and polybutylene glycol, and further includes the monoalkylether of the polyalkylene glycol.

In some embodiments of the present invention, the polyalkylene glycol polymer is a lower alkyl polyalkylene glycol moiety such as a polyethylene glycol moiety (PEG), a polypropylene glycol moiety, or a polybutylene glycol moiety. PEG has the formula —HO(CH2CH2O)nH, where n can range from about 1 to about 4000 or more. In some embodiments, n is 1 to 100, and in other embodiments, n is 5 to 30. PEG can range from an average molecular weight of about 1 to about 22,00. For example, an average molecular weight of about 300 can correspond to n is 5, an average molecular weight of about 2,300 can correspond to n is 50, an average molecular weight of about 13,300 can correspond to n is 300 and an average molecular weight of about 22,000 can correspond to n is 500. In some embodiments, the PEG moiety can be linear or branched. In further embodiments, PEG can be attached to groups such as hydroxyl, alkyl, aryl, acyl or ester. In some embodiments, PEG can be an alkoxy PEG, such as methoxy-PEG (or mPEG), where one terminus is a relatively inert alkoxy group, while the other terminus is a hydroxyl group.

PEG can be readily synthesized or is a commercially available product that can be readily obtained.

According to some embodiments of the present invention, the pegylated compounds of the present invention can be water soluble, soluble in isopropyl alcohol (IPA), ethanol (EtOH), dimethyl sulfoxide (DMSO) and methanol (MeOH), less sensitive to UV light than a non-pegylated counterpart and/or economical to synthesize.

A suitable thiazolium compound of the present invention can be pegylated at least four sites and/or can be pegylated in many differing PEG lengths and molecular weights. In some embodiments, the PEG moiety is PEG₂₀₀ through PEG₅₀₀₀. Pegylated compounds of the present invention can further exhibit improved solubility, enhanced bioavailability, improved stability, lower toxicity, decreased degradation and chemical sensitivities and/or increased conjugation potential to like molecules and other drug molecules.

Medical Uses

Compounds of the present invention have been found to inhibit one or more of the enzymes 5-lipoxygenase, cyclooxygenase, and lyso-PAF: acetyl-CoA acetyltransferase. Additionally, this series of thiazolium derivative were found to inhibit the expression of adhesion molecules on human umbilical endothelial cell monolayers at low concentrations and, are therefore, indicative of utility in treating inflammations, infections and immune disorders.

Examples of inflammatory conditions, infectious conditions or immune disorders are those of the lungs, throat, mouth, joints, eyes, nose, bowel, and skin; particularly those associated with the infiltration of leucocytes into inflamed tissue. Conditions of the lung include asthma, adult respiratory distress syndrome, bronchitis, chronic obstructive pulmonary disease and cystic fibrosis, which may additionally or alternatively involve the bowel or other tissues. Conditions of the throat include laryngitis and orophoryngeal mucositis. Conditions of the mouth include gingivitis and periodontitis. Conditions of the joints include rheumatoid arthritis, rheumatoid spondylitis, osteoarthritis, gouty arthritis and other arthritic conditions. Inflammatory eye conditions include uveitis (including iritis) and conjunctivitis. Inflammatory nose conditions include rhinitis and chronic rhinosinusitis. Inflammatory bowel conditions include Crohn's disease, ulcerative colitis and distal proctitis. Skin diseases include those associated with cell proliferation, such as psoriasis, eczema and dermatitis (whether or not of allergic origin). Other inflammatory conditions and immune disorders include tissue necrosis in chronic inflammation.

Additionally, the present invention provides a method for the prophylaxis or treatment of an inflammatory condition or immune disorder in a mammal, such as a human, which comprises administration of a therapeutically effective amount of a compound of formula I, or a pharmaceutically-acceptable solvate thereof.

In another embodiment of the present invention, there is also provided a compound of formula I, or a pharmaceutically acceptable solvate thereof for use in medical therapy; particularly, for use in the prophylaxis or treatment of an inflammatory condition or immune disorder in a mammal, such as a human.

Additionally, the compounds of formula I were found to have anti-infective activity against certain bacteria, yeast and fungi. Such activity suggests utility for the treatment of topical bacterial, yeast and fungal infections with the compound of formula I. Such infections include Staphylococcus aureus and Streptococcus strains, e.g. pyogenes as well as the yeast strains Candida albicans, Candida tropicalis and Saccharomyces cervisciae and also include the following fungal strains: Cryptococcus neoformans, Aspergillus fumigatus, Aspergillus flavus, Rhizopus arrihizus, Fusarium solani, Microsporidium canis, Microsporidium gypseum, Trichophyton equinium, Trichophyton mentagrophyt, Trichophyton rubrum and Epidermophyton floccsum.

The amount of a compound of formula I or pharmaceutically acceptable solvate thereof, which is required to achieve the desired biological effect will depend on a number of factors such as the use for which it is intended, the means of administration, and the recipient. A typical daily dose for the treatment of septic shock, for instance, may be expected to lie in the range of about 0.005 mg/kg to 100 mg/kg, and in some embodiments, about 0.05 to 50 mg/kg, and in other embodiments, about 0.5 to 20 mg/kg. This dose may be administered as a single unit dose or as several separate unit doses or as a continuous infusion. An intravenous dose may be expected to lie in the range of about 0.0025 mg/kg to 50 mg/kg and would typically be administered as an infusion. Similar dosages would be applicable for the treatment of other disease states. For administration to the lungs of a subject by aerosol an amount of the compound should be used sufficient to achieve concentrations on the airway surface liquid of the subject of about 2 to 1000 mu mol.

Thus, in another aspect of the present invention, there are provided pharmaceutical compositions comprising, as an active ingredient, a compound of formula I or a pharmaceutically acceptable salt or solvate thereof, together with at least one pharmaceutical carrier or recipient. These pharmaceutical compositions may be used in the prophylaxis and treatment of inflammatory conditions, infectious conditions, and immune disorders. The carrier can be pharmaceutically acceptable to the recipient and compatible with, i.e. not have a deleterious effect upon, the other ingredients in the composition. The carrier may be a solid or liquid and is preferably formulated as a unit dose formulation, for example, a tablet which may contain from 0.05 to 95% by weight of the active ingredients. If desired, other physiologically active ingredients may also be incorporated in the pharmaceutical compositions of the invention.

Possible formulations include those suitable for oral, buccal, rectal, topical including dermal, intranasal and inhalation administration. Most suitable means of administration for a particular patient will depend on the nature and severity of the condition being treated and on the nature of the active compound, but where possible, topical administration would be preferred for treatment of topical dermatitis, for instance. For the treatment of a condition such as asthma, however, inhalation, would be the preferred route of administration.

Formulations suitable for oral administration may be provided as discrete units, such as tablets, capsules, cachets, lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically a flavored base, such as sugar and acacia or tragacanth and pastilles comprising the active compound in an inert base, such as gelatin and glycerin or sucrose acacia.

Formulations suitable for rectal administration can be provided as unit-dose suppositories comprising the active ingredient in one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, DMSO and combinations thereof. The active ingredient is typically present in such formulations at a concentration of from 0.1 to 15% w/w.

Moreover, formulations suitable for topical administration include those for medical use and use in personal care, hygiene (e.g., soaps, skin creams and/or lotions, soaps, cleansers, shampoos, wipes, towelettes, gels, etc.) and/or cosmetics. Topical compositions can include the active agents with vitamin E, vitamin A, conjugated linoleic acid, and essential fatty acids. The topical compositions disclosed herein are suitable for topical application to mammalian skin. The compositions include a safe and effective amount of the active agents, and a cosmetically and/or pharmaceutically acceptable topical carrier. The phrase “cosmetically acceptable carrier”, as used herein, means any substantially non-toxic carrier suitable for topical administration to the skin, which has good aesthetic properties, and is compatible with the active agent of the present invention. By “compatible” it is meant that the active agent will remain stable and retain substantial activity therein. The carrier can be in a wide variety of forms, such as sprays, emulsions, mousses, liquids, creams, oils, lotions, ointments, gels and solids.

Suitable pharmaceutically acceptable topical carriers include, but are not limited to, water, glycerol, alcohol, propylene glycol, fatty alcohols, triglycerides, fatty acid esters, and mineral oils. Suitable topical cosmetically acceptable carriers include, but are not limited to, water, petroleum jelly, petrolatum, mineral oil, vegetable oil, animal oil, organic and inorganic waxes, such as microcrystalline, paraffin and ozocerite wax, natural polymers, such as xanthanes, gelatin, cellulose, collagen, starch or gum arabic, synthetic polymers, alcohols, polyols, and the like. The pharmaceutically and/or cosmetically-acceptable carrier can be substantially miscible in water. Such water miscible carrier compositions can also include sustained or delayed release carriers, such as liposomes, microsponges, microspheres or microcapsules, aqueous based ointments, water-in-oil or oil-in-water emulsions, gels and the like.

Formulations of the invention may be prepared by any suitable method, typically by uniformly and intimately admixing the active compound with liquids or finely divided solid carriers or both, in the required proportions and then, if necessary, shaping the resulting mixture into the desired shape.

For example a tablet may be prepared by compressing an intimate mixture comprising a powder or granules of the active ingredient and one or more optional ingredients, such as a binder, lubricant, inert diluent, or surface active dispersing agent, or by molding an intimate mixture of powdered active ingredient and inert liquid diluent.

Aqueous solutions are typically prepared by dissolving the active ingredient in saline to which cyclodextrin has been added.

Suitable formulations for administration by inhalation include fine particle dusts or mists which may be generated by means of various types of metered dose pressurized aerosols, nebulizers, or insufflators.

For pulmonary administration via the mouth, the particle size of the powder or droplets is typically in the range of about 0.5 to 10 μm, and in some embodiments, about 1-5 μm, to ensure delivery into the bronchial tree. For nasal administration, a particle size in the range of about 10 to 500 μm can be employed to ensure retention in the nasal cavity.

Metered dose inhalers are pressurized aerosol dispensers, typically containing a suspension or solution formulation of the active ingredient in a liquefied propellant. During use, these devices discharge the formulation through a valve adapted to deliver a metered volume, typically from about 10 to 150 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol as well as fatty acid surfactants, such as oleic acid or sorbitan trioleate, anti-oxidants and suitable flavoring agents.

Nebulizers are commercially available devices that transform solutions or suspensions of the active ingredient into a therapeutic aerosol mist either by means of acceleration of a compressed gas typically air or oxygen, through a narrow venturi orifice, or by means of ultrasonic agitation. Suitable formulations for use in nebulizers consist of the active ingredient in a liquid carrier and comprising up to about 40% w/w of the formulation, and in some embodiments, less than about 20% w/w. The carrier can be water or a dilute aqueous alcoholic solution, and can be made isotonic with body fluids by the addition of, for example, sodium chloride. Optional additives include preservatives if the formulation is not prepared sterile, for example, methyl hydroxy-benzoate, anti-oxidants, flavoring agents, volatile oils, buffering agents and surfactants.

Suitable formulations for administration by insufflation include finely comminuted powders which may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from about 0.1 to 100 w/w of the formulation.

Therefore, according to a further aspect of the present invention, there is provided the use of a compound of formula (I) or a pharmaceutically acceptable solvate thereof in the preparation of a medicament for the prophylaxis or treatment of an inflammatory condition or immune disorder.

Further, the present invention can provide microcapsules having an ultraviolet absorber and as required an organic solvent enclosed therein, which have capsule wall film of synthetic resin and mean particle size of about 0.1 to 3 μm. These absorbers (ultraviolet absorbers or blockers) may block or inhibit ultraviolet rays.

The following are examples of ultraviolet absorbers (or ultraviolet blockers) that may be used in the present invention.

Phenyl salicylate, p-tert-butylphenyl salicylate, p-octylphenyl salicylate and like salicylic acid type ultraviolet absorbers; 2,4-dihydroxybenzophenone, 2-hydroxy-4-methoxybenzophenone, 2-hydroxy-4-octyloxybenzophenone, 2-hydroxy-4-dodecyloxybenzophenone, 2,2′-dihydroxy-4-methoxybenzophenone, 2,2,′-dihydroxy-4,4′-dimethoxybenzophenone, 2-hydroxy-4-methoxy-5-sulfobenzophenone and like benzophenone type ultraviolet absorbers; 2-ethylhexyl 2-cyano-3,3-diphenyl-acrylate, ethyl 2-cyano-3,3-diphenylacrylate and like cyanoacrylate type ultraviolet absorbers; bis(2,2,6,6-tetramethyl-4-piperidyl)sebacate, bis(2,2,6,6-tetramethyl-4-piperidyl)succinate, bis(1,2,2,6,6-pentamethyl-4-piperidyl) 2-(3′,5-di-tert-butyl-4-hydroxybenzyl)-2-n-butyl malonate and like hindered amine type ultraviolet absorbers; 2-(2′-hydroxyphenyl)benzotriazole, 2-(2′-hydroxy-5′-methylphenyl)benzotriazole, 2-(2′-hydroxy-5-tert-butylphenyl)benzotriazole, 2-(2′-hydroxy-3′,5′-di-tert-butylphenyl)benzotriazole, 2-(2′-hydroxy-3′-tert-butyl-5′-methylphenyl)-5-chlorobenzotriazole, 2-(2′-hydroxy-3′,5′-di-tert-butylphenyl)-5-chlorobenzotriazole, 2-(2′-hydroxy-3′,5′-di-tert-butylphenyl)-5-tert-butylbenzotriazole, 2-(2′-hydroxy-3′,5′-di-tert-amylphenyl)benzotriazole, 2-(2′-hydroxy-3′,5′-di-tert-amylphenyl)-5-tert-amylbenzotriazole, 2-(2′-hydroxy-3′,5′-di-tert-amylphenyl)-5-methoxybenzotriazole, 2-[2′-hydroxy-3′-(3″,4″,5″,6″-tetrahydrophthalimido-methyl)-5′-methylphenyl]benzotriazole, 2-(2′-hydroxy-5′-tert-octylphenyl)benzotriazole, 2-(2′-hydroxy-3′-sec-butyl-5′-tert-butylphenyl)benzotriazole, 2-(2′-hydroxy-3′-tert-amyl-5′-phenoxyphenyl)-5-methylbenzotriazole, 2-(2′-hydroxy-5′-n-dodecylphenyl)benzotriazole, 2-(2′-hydroxy-5′-sec-octyloxyphenyl)-5-phenylbenzotriazole, 2-(2′-hydroxy-3′-tert-amyl-5′-phenylphenyl)-5-methoxybenzotriazole, 2-[2′-hydroxy-3′,5′-bis(α,α-dimethylbenzyl)phenyl]benzotriazole and like benzotriazole type ultraviolet absorbers which are solid at ordinary temperature; 2-(2′-Hydroxy-3′-dodecyl-5′-methylphenyl)-benzotriazole, 2-(2′-hydroxy-3′-undecyl-5′-methylphenyl)-benzotriazole, 2-(2′-hydroxy-3′-tridecyl-5′-methylphenyl)-benzotriazole, 2-(2′-hydroxy-3′-tetradecyl-5′-methylphenyl)-benzotriazole, 2-(2′-hydroxy-3′-pentadecyl-5′-methylphenyl)-benzotriazole, 2-(2′-hydroxy-3′-hexadecyl-5′-methylphenyl)-benzotriazole, 2-[2′-hydroxy-4′-(2″-ethylhexyl)oxyphenyl]-benzotriazole, 2-[2′-hydroxy-4′-(2″-ethylheptyl)oxyphenyl]-benzotriazole, 2-[2′-hydroxy-4′-(2″-ethyloctyl)oxyphenyl]-benzotriazole, 2-[2′-hydroxy-4′-(2″-propyloctyl)oxyphenyl]-benzotriazole, 2-[2′-hydroxy-4′-(2″-propylheptyl)oxyphenyl]-benzotriazole, 2-[2′-hydroxy-4′-(2″-propylhexyl)oxyphenyl]-benzotriazole, 2-[2′-hydroxy-4′-(1″-ethylhexyl)oxyphenyl]-benzotriazole, 2-[2′-hydroxy-4′-(1″-ethylheptyl)oxyphenyl]-benzotriazole, 2-[2′-hydroxy-4′-(1″-ethyloctyl)oxyphenyl]-benzotriazole, 2-[2′-hydroxy-4′-(1 propyloctyl)oxyphenyl]-benzotriazole, 2-[2′-hydroxy-4′-(1″-propylheptyl)oxyphenyl]-benzotriazole, 2-[2′-hydroxy-4′-(1″-propylhexyl)oxyphenyl]-benzotriazole, 2-(2′-hydroxy-3′-sec-butyl-5′-tert-butylphenyl-5-n-butylbenzotriazole, 2-(2′-hydroxy-3′-sec-butyl-5′-tert-butylphenyl)-5-tert-pentyl-benzotriazole, 2-(2′-hydroxy-3′-sec-butyl-5′-tert-butylphenyl)-5-n-pentyl-benzotriazole, 2-(2′-hydroxy-3′-sec-butyl-5′-tert-pentylphenyl)-5-tert-butylbenzotriazole, 2-(2′-hydroxy-3″-sec-butyl-5′-tert-pentylphenyl)-5-n-butylbenzotriazole, 2-(2′-hydroxy-3′,5′-di-tert-butylphenyl)-5-sec-butylbenzotriazole, 2-(2′-hydroxy-3′,5′-di-tert-pentylphenyl)-5-sec-butylbenzotriazole, 2-(2′-hydroxy-3′-tert-butyl-5′-tert-pentylphenyl)-5-sec-butylbenzotriazole, 2-(2′-hydroxy-3′,5′-di-sec-butylphenyl)-5-chlorobenzotriazole, 2-(2′-hydroxy-3′,5′-di-sec-butylphenyl)-5-methoxybenzotriazole, 2-(2′-hydroxy-3′,5′-di-sec-butylphenyl)-5-tert-butylbenzotriazole, 2-(2′-hydroxy-3′,5′-di-sec-butylphenyl)-5-n-butylbenzotriazole, octyl 5-tert-butyl-3-(5-chloro-2H-benzotriazole-2-yl)-4-hydroxybenzene-propionate, condensate of methyl 3-[3-tert-butyl-5-(2H-benzotriazole-2-yl)-4-hydroxyphenyl]propionate and polyethylene glycol (molecular weight: about 300) and like benzotriazole type ultraviolet absorbers which are liquid at ordinary temperature. Of course, the ultraviolet absorber is not limited to thereabove and can be used as required in a mixture of at least two of them.

Although the amount of ultraviolet absorber to be used is not limited specifically, the amount can be adjusted to about 10 to 500 parts by weight, and generally from about 20 to 250 parts by weight.

The microcapsules for use in the present invention can be prepared by various known methods. They are prepared generally by emulsifying and dispersing the core material (oily liquid) comprising an ultraviolet absorber and, if necessary, an organic solvent in an aqueous medium, and forming a wall film of high-molecular-weight substance around the resulting oily droplets.

Examples of useful high-molecular-weight substances for forming the wall film of microcapsules are polyurethane resin, polyurea resin, polyamide resin, polyester resin, polycarbonate resin, aminoaldehyde resin, melamine resin, polystyrene resin, styrene-acrylate copolymer resin, styrene-methacrylate copolymer resin, gelatin, polyvinyl alcohol, etc. Especially, microcapsules having a wall film of a synthetic resin, particularly polyurea resin, polyurethane resin and aminoaldehyde resin among other resins have excellent retainability of an ultraviolet absorber and high heat resistance and accordingly exhibit the outstanding additional effect to serve the function of a pigment which is to be incorporated in the protective layer for preventing sticking to the thermal head. Moreover, microcapsules having a wall film of polyurea resin or polyurethane resin are lower in refractive index than microcapsules with wall films of other materials and usual pigments, are spherical in shape and are therefore usable favorably because even if present in a large quantity in the protective layer, they are unlikely to reduce the density of record images (so-called whitening) owing to irregular reflection of light. Further, polyurea resin and polyurethane resin are more elastic than aminoaldehyde resin and therefore polyirea resin and polyurethane resin are generally used as a wall film for microcapsules which are used under a condition of high pressure. On the other hand, microcapsules having a wall film made from aminoaldehyde resin have a merit that the wall film can be controlled in thickness without depending on particle size of emulsion because the microcapsules can be prepared by adding a wall-forming material after emulsification of a core material.

The present invention may also include organic solvent together with an ultraviolet absorber. The organic solvent is not particularly limited and various hydrophobic solvents can be used which are used in a field of pressure sensitive manifold papers. Examples of organic solvents are tricresyl phosphate, octyldiphenyl phosphate and like phosphates, dibutyl phthalate, dioctyl phthalate and like phthalates, butyl oleate and like carboxylates, various fatty acid amides, diethylene glycol dibenzoate, monoisopropylnaphthalene, diisopropylnaphthalene and like alkylated naphthalenes, 1-methyl-1-phenyl-1-tolylmethane, 1-methyl-1-phenyl-1-xylylmethane, 1-phenyl-1-tolylmethane and like alkylated benzenes, isopropylbiphenyl and like alkylated biphenyls, trimethylolpropane triacrylate and like acrylates, ester of polyols and unsaturated carboxylic acids, chlorinated paraffin and kerosene. These solvents can be used individually or in a mixture of at least two of them. Among these hydrophobic media having a high boiling point, tricresyl phosphate and 1-phenyl-1-tolylmethane are desirable since they exhibit high solubility in connection with the ultraviolet absorber to be used in the present invention. Generally, the lower the viscosity of the core material, the smaller is the particle size resulting from emulsification and the narrower is the particle size distribution, so that a solvent having a low boiling point is conjointly usable to lower the viscosity of the core material. Examples of such solvents having a low boiling point are ethyl acetate, butyl acetate, methylene chloride, etc.

The amount of organic solvent to be used should be suitably adjusted according to the kind and amount of ultraviolet absorber to be used and the kind of organic solvent and is not limited specifically. For example, in case of using an ultraviolet absorber which is liquid at ordinary temperature, an organic solvent is not necessarily used. However, in case of using an ultraviolet absorber which is solid at ordinary temperature, since it is desired that the ultraviolet absorber be in a fully dissolved state in the microcapsules, the amount of organic solvent, for example in case of microcapsules of polyurea resin or polyurethane resin, is adjusted generally from about 10 to 60 wt. %, or from about 20 to 60 wt. %, based on the combined amount of organic solvent, ultraviolet absorber and wall-forming material. Further, in case of microcapsules of aminoaldehyde resin, the amount of organic solvent is adjusted to about 50 to 2000% by weight, generally from about 100 to 1000% by weight of ultraviolet absorber.

Additionally, an absorber may be utilized. An absorber should be selected which reduces the sensitivity of the microcapsule in those portions of its spectral sensitivity range which interfere with the exposure of microcapsules at other wavelengths (its inactive range) without overly reducing the sensitivity of the microcapsule in those portions of the spectral sensitivity range in which the microcapsule is intended to be exposed (its active range). In some cases it may be necessary to balance the absorption characteristics of the absorber in the active range and the inactive range to achieve optimum exposure characteristics. Generally absorbers having an extinction coefficient greater than about 100/M cm in the inactive range and less than about 100,000/M cm in the active range of the microcapsule are used. When the absorber is directly incorporated into the photosensitive composition, ideally, it should not inhibit free radical polymerization, and it should not generate free radicals upon exposure.

The absorbers (ultraviolet absorbers or blockers) used in the present invention can be selected from among those absorbers, which are known in the photographic art. Examples of such compounds include dyes conventionally used as silver halide sensitizing dyes in color photography (e.g., cyanine, merocyanine, hemicyanine and styryl dyes) and ultraviolet absorbers. A number of colored dyes, which absorb outside the desired sensitivity range of the microcapsules and do not absorb heavily within the range could also be used as absorbers in the present invention. Among these, Sudan I, Sudan II, Sudan III, Sudan Orange G, Oil Red O, Oil Blue N, and Fast Garnet GBC are examples of potentially useful compounds.

Additionally ultraviolet absorbers (or ultraviolet blockers) that may be desirable include those selected from hydroxybenzophenones, hydroxyphenylbenzo-triazoles and formamidines. The absorbers may be used alone or in combination to achieve the spectral sensitivity characteristics that are desired.

Representative examples of useful hydroxybenzophenones are 2-hydroxy-4-n-octoxybenzophenone (UV-CHEK AM-300 from Ferro Chemical Division, Mark 1413 from Argus Chemical Division, Witco Chem. Corp., and Cyasorb UV-531 Light Absorber from American Cyanamid), 4-dodecyl-2-hydroxybenzophenone (Eastman Inhibitor DOBP from Eastman Kodak), 2-hydroxy-4-methoxybenzophenone (Cyasorb UV-9 Light Absorber from American Cyanamid), and 2,2′-dihydroxy-4-methoxybenzophenone (Cyasorb UV-24 Light Absorber from American Cyanamid). Representative examples of useful hydroxybenzophenyl benzotriazoles are 2-(2′-hydroxy-5′-methylphenyl)benzotriazole (Tinuvin P from Ciba-Geigy Additives Dept.), 2-(3′,5′-ditert-butyl-2′hydroxyphenyl)-5-chlorobenzotriazole (Tinuvin 327 from Ciba-Geigy), and 2-(2-hydroxy-5-t-octylphenyl)benzotriazole (Cyasorb UV-5411 Light Absorber from American Cyanamid). Representative examples of useful formamidines are described in U.S. Pat. No. 4,021,471 and include N-(p-ethoxy-carbonylphenyl)-N′-ethyl-N′-phenylformamidine (Givsorb UV-2 from Givaudan Corp.). The optimum absorber and concentration of absorber for a particular application depends on both the absorption maximum and extinction coefficient of the absorber candidates and the spectral sensitivity characteristics of the associated photoinitiators.

Additionally, the microcapsules, photosensitive compositions, image-forming agents, developers, and development techniques described in U.S. Pat. Nos. 4,399,209 and 4,440,846, the contents of which are incorporated and may be used in the present invention.

Agricultural Uses

The compounds according to the present invention are also particularly effective against powdery mildews and rusts, pyrenophora, rhynchosporium, tapesia, fusarium and leptosphaeria fungi, in particular against pathogens of monocotyledonous plants such as cereals, including wheat and barley. They are furthermore particularly effective against downy mildew species, powdery mildews, leaf spot diseases and rusts in dicotyledonous plants.

The amount of the compounds of the invention to be applied, will depend on various factors such as the compound employed, the subject of the treatment (substrate, plant, soil, seed), the type of treatment (e.g. spraying, dusting, seed dressing), the purpose of the treatment (prophylactic or therapeutic), the type of fungi and/or bacteria to be treated and the application time.

The fungicidal and/or bactericidal combinations are of particular interest for controlling a large number of fungi and/or bacteria in various crops or their seeds, especially wheat, rye, barley, oats, rice, maize, lawns, cotton, soybeans, coffee, sugarcane, fruit and ornamentals in horticulture and viticulture, in vegetables such as cucumbers, beans and cucurbits, and in field crops such as potatoes, peanuts, tobacco and sugarbeets.

The combinations are applied by treating the fungi and/or bacteria or the seeds, plants or materials threatened by fungus attack, or the soil with a fungicidally and/or bacterially effective amount of the active ingredients.

The agents may be applied before or after infection of the materials, plants or seeds by the fungi and/or bacteria.

When applied to the plants, the compound of formula (I) can be applied at a rate of about 25 to 250 g/ha, generally from about 50 to 150 g/ha, e.g. about 75, 100, 125 or 150 g/ha, in association with about 20 to 2000 g/ha, generally from about 20 to 1000 g/ha.

In agricultural practice the application rates of the combination depend on the type of effect desired, and range from about 0.02 to 3 kg of active ingredient per hectare.

When the active ingredients are used for treating seeds, rates of abut 0.001 to 50 g a.i. per kg, and generally from about 0.01 to 10 g per kg of seed are generally sufficient.

The composition of the invention can be employed in any conventional form, for example in the form of a twin pack, an instant granulate, a flowable formulation, an emulsion concentrate or a wettable powder or surfactant (such as sodium lauryl sulfate and sodium lauryl sulfate salts), in combination with agriculturally acceptable adjuvants. Such compositions may be produced in conventional manner, e.g. by mixing the active ingredients with appropriate adjuvants (diluents or solvents and optionally other formulating ingredients such as surfactants). Also conventional slow release formulations may be employed where long lasting efficacy is intended.

Particularly formulations to be applied in spraying forms such as water dispersible concentrates or wettable powders may contain surfactants such as wetting and dispersing agents, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from about 0.01 to 90% by weight of active agent, from about 0 to 20% agriculturally acceptable surfactant and about 10 to 99.99% solid or liquid adjuvant(s), the active agent consisting of at least the compound of formula I, and optionally other active agents, particularly microbides or conservatives or the like. Concentrated forms of compositions generally contain in between about 2 and 80%, generally from between about 5 and 70% by weight of active agent. Application forms of formulation may for example contain from 0.01 to 20% by weight, generally from about 0.01 to 5% by weight of active agent. Whereas commercial products will generally be formulated as concentrates, the end user will normally employ dilute formulations.

Additionally, the color of the present compounds may be removed by a type of “bleaching”. It is well recognized in the art (cf. for instance B. C. Saunders et al., Peroxidase, London, 1964, p. 10 ff.) that peroxidases act on various amino and phenolic compounds resulting in the production of a color. In view of this, it must be considered surprising that peroxidases (and certain oxidases) may also exert an effect on colored substances in solution such that dye transfer is inhibited. While the mechanism governing the ability of these enzymes to effect dye transfer inhibition has not yet been elucidated, it is currently believed that the enzymes act by reducing hydrogen peroxide or molecular oxygen and oxidizing the colored substance (donor substrate) dissolved or dispersed in the wash liquor, thereby either generating a colorless substance or providing a substance which is not adsorbed to the fabric or building material.

Additionally, a liquid composition of matter according to the present invention may be formed and may be mixed with and/or diluted by an excipient. When the excipient serves as a diluent, it may be a solid, semi-solid, or liquid material which acts as a vehicle, carrier, or medium for the composition of matter. Various suitable excipients will be understood by those skilled in the art and may be found in the National Formulary, 19: 2404-2406 (2000), the disclosure of pages 2404 to 2406 being incorporated by reference herein in their entirety. Excipients can include butanedioal and EDTA. Examples of suitable excipients include, but are not limited to, starches, gum arabic, calcium silicate, microcrystalline cellulose, methacrylates, shellac, polyvinylpyrrolidone, cellulose, water, syrup, and methylcellulose. An aqueous medium may include an active ingredient or ingredients, a quantity of one or more surfactants sufficient to dissolve or suspend said active ingredients uniformly throughout the medium and other manufacturing additives as known to the art. The latter include granulating-binding agents such as gelatin; natural gums, such as acacia, tragacanth; starches, sodium alginate, sugars, polyvinylpyrrolidone; cellulose derivatives such as hydroxypropylmethylcellulose, polyvinyloxoazolidones; pharmaceutical fillers such as lactose, microcrystalline cellulose, dicalcium phosphate, tricalcium phosphate, calcium sulfate, dextrose, mannitol, sucrose; tabletting lubricants if needed such as calcium and magnesium stearate, stearic acid, talc, sterotex (alkaline stearate). The term “aqueous medium” for one ingredient of one of the embodiments of the invention is used within the custom of the art. Primarily, it connotes a water medium, with added water-miscible solvents such as isopropanol or ethanol when needed, to support the active ingredient.

Industrial Uses

In addition to the uses and formulations described above, the compounds of the present invention may be provided in an aerosol or non-aerosol spray product that may be applied to surfaces in residential areas, medical facilities, commercial areas or vehicles, aircrafts, trains, buses, etc. where microbial growth is present or likely to exist. For example, the compounds of the present invention may be applied in bathrooms, kitchens, garages, pool areas, etc. of homes, hospitals, hotels, daycares, communal living facilities, restaurants, airplanes, buses, trains etc., where applicable. The aerosol spray, whether formed from solid or liquid particles, can be produced by an aerosol generator. Any suitable propellant may be used in carrying out the present invention.

Further, the compounds of the present invention may also be used to treat all areas where microorganisms described herein, such as algae, molds, fungi and bacteria, are grown. Examples include, but are not limited to wood, air ducts, lumber, floorings, decks, buoys, seawalls, retaining walls, docks, pilings, watercrafts, boats, pipes, stucco, tiles, paint, insulation, roofs, roofing materials, building materials, metal, concrete and cement-based materials, plasters, asphalts, ceramics, stucco, sheetrock, grout, caulking, mortar, plastics, foam, glass, carpets, wallpaper, cloth, computer parts, food packaging, paper products, medical devices, petroleum processing, oil and natural gas extraction, metal working fluids, fasteners, adhesives, sealants, recreational water bodies, such as swimming pools, saunas, hot tubs, whirlpools, jacuzzis and spas, etc., and surfaces thereof, wall coverings, siding materials, flooring, filtration systems, cooling towers and substrates, etc.

Products such as wood, floorings, tiles, paint, insulation, roofs, roofing materials, other building materials, ceramics, plastics, foam, glass, carpets, wallpaper, cloth, computer parts, etc. that come in contact with humans and animals provide an opportunity to introduce various pathogens to the subject. Accordingly, treatment of such products with the compounds of formula I may present a mechanism to reduce microbial infections in humans and animals.

Additionally, marine coatings serve as an application for the antimicrobial compounds of the present invention. When incorporated into paint or coatings on decks, buoys, pilings, the hulls of ships or on metal subsurfaces, etc. in a marine environment, the compounds of formula I may reduce or prevent biological deposits and biological corrosion. As understood by one of ordinary skill in the art, a slime layer only about 1 millimeter thick on a hull can reduce the speed of a vessel by at least about 15 percent and increase fuel costs correspondingly. Heavier deposits can also result in corrosion of the metal itself thereby limiting the life of the coating, requiring premature dry-docking of the vessel. Application of the compounds of formula I may combat these effects.

Petroleum processing and oil and natural gas extraction can use extensive amounts of antimicrobials to prevent or reduce the souring of natural gas, crude oil and water in oil fields. Water is used both in drilling muds to lubricate the drill and as fluid to force crude petroleum from oil-bearing rock. The use of compounds of formula I may facilitate the decontamination process involved in these operations.

Metal working fluids are used at manufacturing facilities to cool and lubricate metal parts being drilled, milled, machined or formed. These fluids are primarily water-based emulsions, although some petroleum-based fluids can also be used. In addition to cooling and lubricating, metal working fluids can also function to flush metal particles from the process surfaces. While water-based fluids are particularly susceptible to microbial growth, petroleum-based fluids can become tainted when microbial growth occurs in any water collecting in the containment system under the oil phase. Microbial contamination can cause noxious odors, decomposition of the lubricating agents, acidity that can be detrimental to machine tool parts, and, in some instances, a limited health hazard during prolonged exposure of workers' skin to the fluid. Accordingly, compounds of formula I may minimize these effects.

Paper products, especially recycled paper, which is even more prone to microbial contamination, and paper coatings, can be treated with compounds of formula I in an effort to prevent microbial growth on surfaces, and thus, prevent the contamination and ultimate spoilage of goods.

Cooling towers, which are an integral part of temperature control systems, can remove chemicals and biological contamination from the air and trap it in the cooling liquid and can rapidly become contaminated with a variety of microorganisms. The presence of slime deposits can reduce heat transfer and increase energy requirements. The occurrence of possible pathogenic organisms is a secondary concern in cooling towers and evaporative condenser systems. Each cooling season, individuals are suspected of developing pulmonary disease due to Legionella pneumophila associated with cooling towers and the cooling process. Compounds of formula I may reduce the pathogens associated with the operation of cooling towers.

Accordingly, embodiments of the present invention further include application of the compounds of the present invention onto various articles of manufacture, substrates and/or materials and/or use in processes listed above as well as incorporation into the products to form an integral part of the material. For example, compounds of the present invention may be coated or sprayed onto and/or incorporated into the substrate forming the medical device, such as a stent, for the prevention of biofilm formation. Compounds of the present invention may be coated onto a cement-based material and/or included in the cement mix during formation of the cement-based material. Lumber may be pressure-treated with the compounds of the present invention and/or soaked with a solution including the compounds. Fabrics may be coated or sprayed or soaked with the compounds of the present invention, or individual strands may be treated prior to the weaving or fabrication process. Other building materials such as wall board, masonite, particle board, etc. may be treated with compounds of the present invention, or the compounds may be added to the slurry or mixture during the fabrication of the materials so that the compounds of the present invention become an integral part of intermediate and final materials. The amount of the compound to be added during the fabrication process can be determined through routine experimentation and in view of government regulations through agencies such as the Environmental Protection Agency (EPA), U.S. Food and Drug Administration (FDA) and U.S. Department of Agriculture (USDA), as well as foreign counterparts.

Factors that can influence the concentrations necessary to combat microorganisms in a swimming pool, hot tub, spa, etc. include, but are not limited to, the number of individuals using the area; frequency of use; frequency with which water is changed; general weather conditions; and types and degree of organic contamination of the water by the users themselves (e.g., suntan lotions and oils) and by various debris. Therefore, laboratory testing and/or confirmatory field testing as conducted by one of skill in the art can be used to ascertain the concentration of the compounds of the present invention to achieve the desired effect.

To combat the growth of microorganisms, a recreational body of water may include from about 0.001 ppm (parts per million) by weight to about 2500 ppm compounds of formula I. In some embodiments, the concentration can be about 1 ppm by weight to 2200 ppm by weight, and in some other embodiments, about 5 to 500 ppm by weight. Further embodiments may include about 5 to 25 ppm by weight compounds of formula I.

Microorganisms and Microbial Infections

In addition to the microorganisms previously discussed, microorganisms that can be affected according to methods of the present invention include, but are not limited to, bacteria, mycobacteria, spirochetes, rickettsia, chlamydia, mycoplasma, algae, fungi, protozoans, viruses, and parasites. Accordingly, methods disclosed herein relate to bacterial, mycobacterial, spirochetal, rickettsial, chlamydial, mycoplasmal, algal, fungal, viral, and parasitic infections.

Further bacterial infections that can be treated using the active agents of the present invention can be caused by bacteria such as gram-negative bacteria. Examples of gram-negative bacteria include, but are not limited to, bacteria of the genera, Salmonella, Escherichia, Klebsiella, Haemophilus, Pseudomonas, Proteus, Neisseria, Vibro, Helicobacter, Brucella, Bordetella, Legionella, Campylobacter, Francisella, Pasteurella, Yersinia, Bartonella, Bacteroides, Streptobacillus, Spirillum and Shigella. Furthermore, bacterial infections that can be treated using the active agents of the present invention can be caused by gram-negative bacteria including, but not limited to, Escherichia coli, Pseudomonas aeruginosa, Neisseria meningitides, Neisseria gonorrhoeae, Salmonella typhimurium, Salmonella entertidis, Klebsiella pneumoniae, Haemophilus influenzae, Haemophilus ducreyi, Proteus mirabilis, Vibro cholera, Helicobacter pylori, Brucella abortis, Brucella melitensis, Brucella suis, Bordetella pertussis, Bordetella parapertussis, Legionella pneumophila, Campylobacter fetus, Campylobacter jejuni, Francisella tularensis, Pasteurella multocida, Yersinia pestis, Bartonella bacilliformis, Bacteroides fragilis, Bartonella henselae, Streptobacillus moniliformis, Spirillum minus and Shigella dysenteriae.

Bacterial infections that can be treated using the active agents of the present invention can also be caused by bacteria such as gram-positive bacteria. Examples of gram-positive bacteria include, but are not limited to, bacteria of the genera Listeria, Staphylococcus, Streptococcus, Bacillus, Corynebacterium, Peptostreptococcus, and Clostridium. Furthermore, bacterial infections that can be treated using the active agents of the present invention can be caused by gram-positive bacteria including, but not limited to, Listeria monocytogenes, Staphylococcus aureus, Streptococcus pyogenes, Streptococcus pneumoniae, Bacillus cereus, Bacillus anthracis, Clostridium botulinum, Clostridium perfringens, Clostridium difficile, Clostridium tetani, Corynebacterium diphtheriae and Peptostreptococcus anaerobius. In some embodiments, the gram-positive bacteria is methicillin-resistant Staphylococcus aureus.

Additional bacterial infections that can be treated using the active agents of the present invention can be caused by bacteria in the genera including, but not limited to, Actinomyces, Propionibacterium, Nocardia and Streptomyces. Furthermore, bacterial infections that can be treated using the active agents of the present invention can be caused by bacteria including, but not limited to, Actinomyces israeli, Actinomyces gerencseriae, Actinomyces viscosus, Actinomyces naeslundii, Propionibacterium propionicus, Nocardia asteroides, Nocardia brasiliensis, Nocardia otitidiscaviarum and Streptomyces somaliensis.

Mycobacterial infections that can be treated by the compounds of the present invention can be caused by mycobacteria belonging to the mycobacteria families including, but not limited to, Mycobacteriaceae. Additionally, mycobacterial infections that can be treated by the compounds of the present invention can be caused by mycobacteria including, but not limited to, Mycobacterium tuberculosis, Mycobacterium leprae, Mycobacterium avium-intracellulare, Mycobacterium kansasii, and Mycobacterium ulcerans.

Spirochetal infections that can be treated using the active agents of the present invention can be caused by spirochetes belonging to the genera including, but not limited to, Treponema, Leptospira, and Borrelia. Additionally, spirochetal infections that can be treated using the active agents of the present invention can be caused by the spirochetes including, but not limited to, Treponema palladium, Treponema pertenue, Treponema carateum, Leptospira interrogans, Borrelia burgdorferi, and Borrelia recurrentis.

Rickettsial infections that can be treated using the active agents of the present invention can be caused by rickettsia belonging to the genera including, but not limited to, Rickettsia, Ehrlichia, Orienta, Bartonella and Coxiella. Furthermore, rickettsial infections that can be treated using the active agents of the present invention can be caused by rickettsia including, but not limited to, Rickettsia rickettsii, Rickettsia akari, Rickettsia prowazekii, Rickettsia typhi, Rickettsia conorii, Rickettsia sibirica, Rickettsia australis, Rickettsia japonica, Ehrlichia chaffeensis, Orienta tsutsugamushi, Bartonella quintana, and Coxiella burni.

Chlamydial infections that can be treated using the active agents of the present invention can be caused by chlamydia belonging to the genera including, but not limited to, Chlamydia. Furthermore, chlamydial infections that can be treated using the active agents of the present invention can be caused by chlamydia including, but not limited to, Chlamydia trachomatis, Chlamydia caviae, Chlamydia pneumoniae, Chlamydia muridarum, Chlamydia psittaci, and Chlamydia pecorum.

Mycoplasmal infections that can be treated using the active agents of the present invention can be caused by mycoplasma belonging to the genera including, but not limited to, Mycoplasma and Ureaplasma. In addition, mycoplasmal infections that can be treated using the active agents of the present invention can be caused by mycoplasma including, but not limited to, Mycoplasma pneumoniae, Mycoplasma hominis, Mycoplasma genitalium, and Ureaplasma urealyticum.

Fungal infections that can be treated using the active agents of the present invention can be caused by fungi belonging to the genera including, but not limited to, Aspergillus, Candida, Cryptococcus, Coccidioides, Tinea, Sporothrix, Blastomyces, Histoplasma, and Pneumocystis. Additionally, fungal infections that can be treated using the active agents of the present invention can be caused by fungi including, but not limited to, Aspergillus fumigatus, Aspergillus flavus, Aspergillus niger, Aspergillus terreus, Aspergillus nidulans, Candida albicans, Coccidioides immitis, Cryptococcus neoformans, Tinea unguium, Tinea corporis, Tinea cruris, Sporothrix schenckii, Blastomyces dermatitidis, Histoplasma capsulatum, and Histoplasma duboisii.

Viral infections that can be treated using the active agents of the present invention can be caused by viruses belonging to the viral families including, but not limited to, Flaviviridae, Arenaviradae, Bunyaviridae, Filoviridae, Poxyiridae, Togaviridae, Paramyxoviridae, Herpesviridae, Picornaviridae, Caliciviridae, Reoviridae, Rhabdoviridae, Papovaviridae, Parvoviridae, Adenoviridae, Hepadnaviridae, Coronaviridae, Retroviridae, and Orthomyxoviridae. Furthermore, viral infections that can be treated using the active agents of the present invention can be caused by the viruses including, but not limited to, Yellow fever virus, St. Louis encephalitis virus, Dengue virus, Hepatitis G virus, Hepatitis C virus, Bovine diarrhea virus, West Nile virus, Japanese B encephalitis virus, Murray Valley encephalitis virus, Central European tick-borne encephalitis virus, Far eastern tick-born encephalitis virus, Kyasanur forest virus, Louping ill virus, Powassan virus, Omsk hemorrhagic fever virus, Kumilinge virus, Absetarov anzalova hypr virus, Ilheus virus, Rocio encephalitis virus, Langat virus, Lymphocytic choriomeningitis virus, Junin virus, Bolivian hemorrhagic fever virus, Lassa fever virus, California encephalitis virus, Hantaan virus, Nairobi sheep disease virus, Bunyamwera virus, Sandfly fever virus, Rift valley fever virus, Crimean-Congo hemorrhagic fever virus, Marburg virus, Ebola virus, Variola virus, Monkeypox virus, Vaccinia virus, Cowpox virus, Orf virus, Pseudocowpox virus, Molluscum contagiosum virus, Yaba monkey tumor virus, Tanapox virus, Raccoonpox virus, Camelpox virus, Mousepox virus, Tanterapox virus, Volepox virus, Buffalopox virus, Rabbitpox virus, Uasin gishu disease virus, Sealpox virus, Bovine papular stomatitis virus, Camel contagious eethyma virus, Chamios contagious eethyma virus, Red squirrel parapox virus, Juncopox virus, Pigeonpox virus, Psittacinepox virus, Quailpox virus, Sparrowpox virus, Starlingpox virus, Peacockpox virus, Penguinpox virus, Mynahpox virus, Sheeppox virus, Goatpox virus, Lumpy skin disease virus, Myxoma virus, Hare fibroma virus, Fibroma virus, Squirrel fibroma virus, Malignant rabbit fibroma virus, Swinepox virus, Yaba-like disease virus, Albatrosspox virus, Cotia virus, Embu virus, Marmosetpox virus, Marsupialpox virus, Mule deer poxvirus virus, Volepox virus, Skunkpox virus, Rubella virus, Eastern equine encephalitis virus, Western equine encephalitis virus, Venezuelan equine encephalitis virus, Sindbis virus, Semliki forest virus, Chikungunya virus, O'nyong-nyong virus, Ross river virus, Parainfluenza virus, Mumps virus, Measles virus (rubeola virus), Respiratory syncytial virus, Herpes simplex virus type 1, Herpes simplex virus type 2, Varicella-zoster virus, Epstein-Barr virus, Cytomegalovirus, Human b-lymphotrophic virus, Human herpesvirus 7, Human herpesvirus 8, Poliovirus, Coxsackie A virus, Coxsackie B virus, ECHOvirus, Rhinovirus, Hepatitis A virus, Mengovirus, ME virus, Encephalomyocarditis (EMC) virus, MM virus, Columbia SK virus, Norwalk agent, Hepatitis E virus, Colorado tick fever virus, Rotavirus, Vesicular stomatitis virus, Rabies virus, Papilloma virus, BK virus, JC virus, B19 virus, Adeno-associated virus, Adenovirus, serotypes 3, 7, 14, 21, Adenovirus, serotypes 11, 21, Adenovirus, Hepatitis B virus, Coronavirus, Human T-cell lymphotrophic virus, Human immunodeficiency virus, Human foamy virus, Influenza viruses, types A, B, C, and Thogotovirus.

Plant viruses include viruses in the following groups: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picornaviridae; Plasmaviridae; Prodoviridae; Polydnaviridae; Potexvirus group; Potyvirus; Poxyiridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and plant virus satellites. Plant viruses further include those in the Genus Tobamovirus, which includes Tobacco mosaic virus (TMV).

Geminiviruses encompass viruses of the Genus Mastrevirus, Genus Curtovirus, and Genus Begomovirus. Exemplary geminiviruses include, but are not limited to, Abutilon Mosaic Virus, Ageratum Yellow Vein Virus, Bhendi Yellow Vein Mosaic virus, Cassaya African Mosaic Virus, Chino del Tomato Virus, Cotton Leaf Crumple Virus, Croton Yellow Vein Mosaic Virus, Dolichos Yellow Mosaic Virus, Horsegram Yellow Mosaic Virus, Jatropha Mosaic virus, Lima Bean Golden Mosaic Virus, Melon Leaf Curl Virus, Mung Bean Yellow Mosaic Virus, Okra Leaf Curl Virus, Pepper Hausteco Virus, Potato Yellow Mosaic Virus, Rhynchosia Mosaic Virus, Squash Leaf Curl Virus, Tobacco Leaf Curl Virus, Tomato Australian Leaf Curl Virus, Tomato Indian Leaf Curl Virus, Tomato Leaf Crumple Virus, Tomato Yellow Leaf Curl Virus, Tomato Yellow Mosaic Virus, Watermelon Chlorotic Stunt Virus, Watermelon Curly Mottle Virus, Bean Distortion Dwarf Virus, Cowpea Golden Mosaic Virus, Lupin Leaf Curl Virus, Solanum Apical Leaf Curling Virus, Soybean Crinkle Leaf Virus, Chloris Striate Mosaic Virus, Digitaria Striate Mosaic Virus, Digitaria Streak Virus, Miscanthus Streak Virus, Panicum Streak Virus, Pasalum Striate Mosaic Virus, Sugarcane Streak Virus, Tobacco Yellow Dwarf Virus, Cassaya Indian Mosaic Virus, Serrano Golden Mosaic Virus, Tomato Golden Mosaic Virus, Cabbage Leaf Curl Virus, Bean Golden Mosaic Virus, Pepper Texas Virus, Tomato Mottle Virus, Euphorbia Mosaic Virus, African Cassaya Mosaic Virus, Bean Calico Mosaic Virus, Wheat Dwarf Virus, Cotton Leaf Curl Virus, Maize Streak Virus, and any other virus designated as a Geminivirus by the International Committee on Taxonomy of Viruses (ICTV).

Badnaviruses are a genus of plant viruses having double-stranded DNA genomes. Specific badnavirus include cacao swollen shoot virus and rice tungro bacilliform virus (RTBV). Most badnavirus have a narrow host range and are transmitted by insect vectors. In the badnaviruses, a single open reading frame (ORF) may encode the movement protein, coat protein, protease and reverse transcriptase; proteolytic processing produces the final products. Exemplary Badnaviruses include, but are not limited to Commelina Yellow Mottle Virus, Banana Streak Virus, Cacao Swollen Shoot Virus, Canna Yellow Mottle Virus, Dioscorea Bacilliform Virus, Kalanchoe Top-Spotting Virus, Piper Yellow Mottle Virus, Rice Tungro Bacilliform Virus, Schefflera Ringspot Virus, Sugarcane Bacilliform Virus, Aucuba Bacilliform Virus, Mimosa Baciliform Virus, Taro Bacilliform Virus, Yucca Bacilliform Virus, Rubus Yellow Net Virus, Sweet Potato Leaf Curl Virus, Yam Internal Brown Spot Virus, and any other virus designated as a Badnavirus by the International Committee on Taxonomy of Viruses (ICTV).

Caulimoviruses have double-stranded circular DNA genomes that replicate through a reverse transcriptase-mediated process, although the virus DNA is not integrated into the host genome. As used herein, Caulimoviruses include but are not limited to Cauliflower Mosaic Virus, Blueberry Red Ringspot Virus, Carnation Etched Ring Virus, Dahlia Mosaic Virus, Figwort Mosaic Virus, Horseradish Latent Virus, Mirabilis Mosaic Virus, Peanut Chlorotic Streak Virus, Soybean Chlorotic Mottle Virus, Strawberry Vein Banding Virus, Thistle Mottle Virus, Aquilegia Necrotic Mosaic Virus, Cestrum Virus, Petunia Vein Clearing Virus, Plantago Virus, Sonchus Mottle Virus, and any other virus designated as a Caulimovirus by the International Committee on Taxonomy of Viruses (ICTV).

The Nanoviruses have single-stranded circular DNA genomes. As used herein, Nanoviruses include but are not limited to Banana Bunchy Top Nanavirus, Coconut Foliar Decay Nanavirus, Faba Bean Necrotic Yellows Nanavirus, Milk Vetch Dwarf Nanavirus, and any other virus designated as a Nanovirus by the International Committee on Taxonomy of Viruses (ICTV).

Protozoans that can be treated using the active agents of the present invention include flagellates, amoebae, sporozoans and ciliates.

Parasitic infections that can be treated using the active agents of the present invention can be caused by parasites belonging to the genera including, but not limited to, Entamoeba, Dientamoeba, Giardia, Balantidium, Trichomonas, Cryptosporidium, Isospora, Plasmodium, Leishmania, Trypanosoma, Babesia, Naegleria, Acanthamoeba, Balamuthia, Enterobius, Strongyloides, Ascaradia, Trichuris, Necator, Ancylostoma, Uncinaria, Onchocerca, Mesocestoides, Echinococcus, Taenia, Diphylobothrium, Hymenolepsis, Moniezia, Dicytocaulus, Dirofilaria, Wuchereria, Brugia, Toxocara, Rhabditida, Spirurida, Dicrocoelium, Clonorchis, Echinostoma, Fasciola, Fascioloides, Opisthorchis, Paragonimus, and Schistosoma. Additionally, parasitic infections that can be treated using the active agents of the present invention can be caused by parasites including, but not limited to, Entamoeba histolytica, Dientamoeba fragilis, Giardia lamblia, Balantidium coli, Trichomonas vaginalis, Cryptosporidium parvum, Isospora belli, Plasmodium malariae, Plasmodium ovale, Plasmodium falciparum, Plasmodium vivax, Leishmania braziliensis, Leishmania donovani, Leishmania tropica, Trypanosoma cruzi, Trypanosoma brucei, Babesia divergens, Babesia microti, Naegleria fowleri, Acanthamoeba culbertsoni, Acanthamoeba polyphaga, Acanthamoeba castellanii, Acanthamoeba astronyxis, Acanthamoeba hatchetti, Acanthamoeba rhysodes, Balamuthia mandrillaris, Enterobius vermicularis, Strongyloides stercoralis, Strongyloides fulleborni, Ascaris lumbricoides, Trichuris trichiura, Necator americanus, Ancylostoma duodenale, Ancylostoma ceylanicum, Ancylostoma braziliense, Ancylostoma caninum, Uncinaria stenocephala, Onchocerca volvulus, Mesocestoides variabilis, Echinococcus granulosus, Taenia solium, Diphylobothrium latum, Hymenolepis nana, Hymenolepis diminuta, Moniezia expansa, Moniezia benedeni, Dicytocaulus viviparous, Dicytocaulus filarial, Dicytocaulus arnfieldi, Dirofilaria repens, Dirofilaria immitis, Wuchereria bancrofti, Brugia malayi, Toxocara canis, Toxocara cati, Dicrocoelium dendriticum, Clonorchis sinensis, Echinostoma, Echinostoma ilocanum, Echinostoma jassyenese, Echinostoma malayanum, Echinostoma caproni, Fasciola hepatica, Fasciola gigantica, Fascioloides magna, Opisthorchis viverrini, Opisthorchis felineus, Opisthorchis sinensis, Paragonimus westermani, Schistosoma japonicum, Schistosoma mansoni, Schistosoma haematobium and Schistosoma haematobium.

Subjects suitable to be treated for non-industrial purposes include, but are not limited to, plant, avian and mammalian subjects. Mammals of the present invention include, but are not limited to, canines, felines, bovines, caprines, equines, ovines, porcines, rodents (e.g., rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and birds in ovo.

The invention can also be carried out on animal subjects, particularly mammalian subjects such as mice, rats, dogs, cats, livestock and horses for veterinary purposes, and for drug screening and drug development purposes.

### EXAMPLES

The present invention is explained in greater detail in the Examples that follow. These examples are intended as illustrative of the invention and are not to be taken are limiting thereof.

In the following Examples, the “active ingredient” may be any compound of formula I as recited above or a pharmaceutically acceptable salt or a solvate thereof.

These compounds can also include the general Formula II

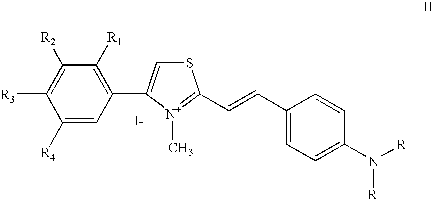

wherein R is a lower alkyl;

wherein R₁ is selected from the group consisting of hydrogen and a lower alkyl;

wherein R₂ is selected from the group consisting of hydrogen and a lower alkyl;

wherein R₃ is selected from the group consisting of hydrogen, alkoxy and a lower alkyl;

wherein R₄ is selected from the group consisting of hydrogen and a lower alkyl; or a solvate thereof.

These compounds can further include Formula III: 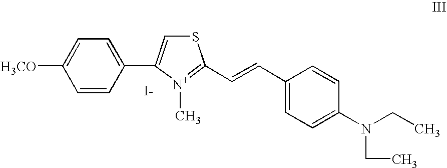
p-Methoxyphenyl methyl ketone is reacted with bromine in a non-polar solvent to produce the corresponding p-methoxyphenacyl bromide. Reaction of this reactive alpha-bromo ketone with commercially available thioacetamide in an protic solvent such as methanol with heat affords the corresponding 2-methyl-4-(4-methoxy)phenylthiazole. N-Alkylation of this thiazole with methyl iodide in aprotic solvents such as dimethylformamide and heat forms the corresponding methiodide product, which is also named either N-methyl-2-methyl-4-(4-methoxyphenyl)thiazolium iodide or 3-methyl-2-methyl-4-(4-methoxyphenyl)thiazolium iodide. Reaction of this thiazolium iodide with 4-(N,N-diethyl)aminobenzaldehyde in a protic solvent such as methanol with a basic catalyst such as piperidine and heat, followed by crystallization, then produces the desired E-2-(N,N-diethylaminostyryl)-3-methyl-4-(4-methoxyphenyl)thiazolium iodide (III), which can also be named E-2-[2-(4-diethylaminophenyl)vinyl]-3-methyl-4-(4-methoxyphenyl)thiazol-3-ium iodide (III);
Formula IV: 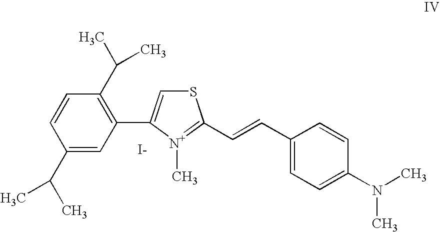
(2,5-Diisopropyl)phenyl methyl ketone is reacted with bromine in a non-polar solvent to produce the corresponding (2,5-diisopropyl)phenacyl bromide. Reaction of this reactive alpha-bromo ketone with commercially available thioacetamide in an protic solvent such as methanol with heat affords the corresponding 2-methyl-4-(2,5-diisopropyl)phenylthiazole. N-Alkylation of this thiazole with methyl iodide in aprotic solvents such as dimethylformamide and heat forms the corresponding methiodide product, which is also named either N-methyl-2-methyl-4-(2,5-diisopropylphenyl)thiazolium iodide or 3-methyl-2-methyl-4-(2,5-diisopropylphenyl)thiazolium iodide. Reaction of this thiazolium iodide with 4-(N,N′-dimethyl)aminobenzaldehyde in a protic solvent such as methanol with a basic catalyst such as piperidine and heat, followed by crystallization, then produces the desired E-2-(N,N′-dimethylaminostyryl)-3-methyl-4-(2,5-diisopropylphenyl)thiazolium iodide (IV) which can also be named E-2-[2-(4-dimethylaminophenyl)vinyl]-4-(2,5-dimethylphenyl)-3-methylthiazol-3-ium iodide (IV); or Formula V: 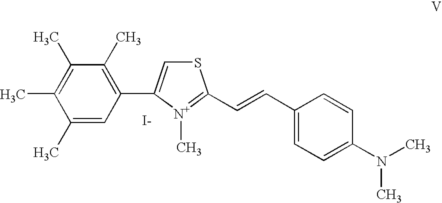
(2,3,4,5-Tetramethyl)phenyl methyl ketone is reacted with bromine in a non-polar solvent to produce the corresponding (2,3,4,5-tetramethyl)phenacyl bromide. Reaction of this reactive alpha-bromo ketone with commercially available thioacetamide in an protic solvent such as methanol with heat affords the corresponding 2-methyl-4-(2,3,4,5-tetramethyl)phenylthiazole. N-Alkylation of this thiazole with methyl iodide in aprotic solvents such as dimethylformamide and heat forms the corresponding methiodide product, which is also named either N-methyl-2-methyl-4-(2,3,4,5-tetramethylphenyl)thiazolium iodide or 3-methyl-2-methyl-4-(2,3,4,5-tetramethylphenyl)thiazolium iodide. Reaction of this thiazolium iodide with 4-(N,N′-dimethyl)aminobenzaldehyde in a protic solvent such as methanol with a basic catalyst such as piperidine and heat, followed by crystallization, then produces the desired E-2-(N,N′-dimethylaminostyryl)-3-methyl-4-(2,3,4,5-tetramethylphenyl)thiazolium iodide (V) which can also be named E-2-[2-(4-dimethylaminophenyl)vinyl]-3-methyl-4-(2,3,4,5-tetramethylphenyl)thiazol-3-ium iodide (V).

Compounds III, IV and V were prepared and were subjected to Anti-Fungal Activity in Plant Relevant Molds In Vitro Microtitre Tests. Table I illustrates the antifungal activities of these compounds.

TABLE 1
Anti-Fungal Activity in Plant Relevant Molds In Vitro Microtitre
Tests

| | | Formula | Formula | Formula |
|---|---|---|---|---|
| | | III | IV | V |
| Organism | Plant | IC90 ppm | | |
| Alternaria solani | Potato | 31 | 8 | 8 |
| Botrytis cinerea | Vegetable | 2 | 2 | 2 |
| Cochliobolus | Corn | 2 | 8 | 8 |
| mijabeanus | | | | |
| Colletotrichum | Mellons | 8 | 8 | 8 |
| lagenarium | | | | |
| Fusarium | Wheat Head | 31 | 125 | 31 |
| culmorum | | | | |
| Phytophthora | Tomato | 2 | 8 | 2 |
| infestans | | | | |
| Pyrenophora teres | Barley | 8 | 8 | 8 |
| Pyricularia oryzae | Rice | 8 | 8 | 8 |
| Rhizoctonia solani | Rice Sheath | 8 | 8 | 8 |
| Septonia tritici | Wheat Leaf | 2 | 2 | 2 |

A stock solution of each compound was prepared in DMSO at a concentration of 10,000 ppm a.i. Further dilutions were prepared with water. The test was conducted at the following concentrations: 125, 31, 8, 21, 0.5 and 0.125 ppm a.i. Spore suspensions of the fungi were prepared. The test was conducted in microtiter plates and for each fungus and each concentration, 3 wells were prepared. Incubation of the inoculated plates was carried out at 18° C. for 7 days. After this time, the optical density of the mycelium developed in each well was measured at 405 nm.

The data produced allowed an assessment of the IC 90 value (the concentration at which the fungal growth was reduced by at least 90% compared to the control).

### Example 2

### Anti-Icam1 Activity in Huvec Assay

Inhibition of Cytokine-Induced Adhesiveness of Endothelial Cells for Neutrophils.

The compounds known above as E-2-(4-dimethylaminostyryl)-4-(2,5-diisopropylphenyl)-3-methylthiazolium iodide (formula IV) and E-2-(4-dimethylaminostyryl)-3-methyl-4-(2,3,4,5-tetramethylphenyl)thiazolium iodide (formula V), as well as E-2-(p-pyrrolidinostyryl)-4-(p-biphenyl)-3-methylthiazolium iodide, and E-2-(4′-diethylaminostyryl)-4-(4″-ethoxyphenyl)-3-methylthiazolium iodide (formula III) all exhibited anti-ICAM1 activity at an IC₅₀ nM of less than 80. Leukocyte adhesion to the vascular endothelium is a critical step in mounting an effective inflammatory or immune response, thereby representing an important therapeutic target for inflammatory or immune disorders. ICAM-1 as well as other cellular adhesion molecules are intimately involved in this step. The above compounds demonstrated anti-adhesive activity in the Human Umbilical Vein Endothelial Cells Assay. In the assay, the HUVE cells were layered and incubated in normal medium. The test compounds were then applied to the layered cells for 1 hour. The cells were washed with medium and then a cell adhesion stimulant (TNF-alpha, IL-1 or LPS) was applied for 1 hour. The HUVE cells were washed with medium and normal human WBCs were applied and incubated for 4 hours. The HUVE cells were washed with medium and the number of WBCs attached to the HUVEC was determined by radio-label. Viability of the WBCs was also determined. A decrease in the number of WBCs on the HUVEC indicates an inhibition of cell adhesion as long as the WBCs are determined to be not damaged.

### Example 3

### Acute Anti-Inflammatory Activity in the 4HR Carrageenan Pleurisy Assay in Rats

The Acute Local Carrageenan Pleuritis Assay in Rats is an in vivo model to determine local acute anti-inflammatory activity of compounds based on inhibition of edema formation and neutrophil mobilization into the pleural cavity. In this assay, male Lewis rats of approximately 200 gms were utilized. A carrageenan solution (400 μg/ml) was prepared in water. The experimental compounds were mixed into the carrageenan solution. The carrageenan +/− compounds was injected intrapleurally 0.25 cc/rat. The rats were sacrificed four hours later. The pleural cavity was opened and measured and the exudate extracted. The pleural cavity was washed with 5 cc EDTA solution to capture pleural cells. The total WBCs in the wash were counted and recording. Next the compound inhibition of exudate volume and inflammatory cell influx wash determined. Every experiment included a positive (prednisolone) and negative control group. The following table illustrates the results of this test.

Table 2

TABLE 2

| | Acute Anti-inflammatory |
|---|---|
| | Activity in Rats |
| Compounds | ED50 mg/rat |
| 2-(4′-diethylaminostyryl)-4- | 0.001 |
| (4″-ethoxyphenyl)-3-methyl- | |
| thiazolium iodide | |
| 4-(4-biphenyl)-3-ethyl-2-[(4- | 0.001 |
| pyrrolidino)styryl]thiazolium | |
| iodide | |
| 2-[2-(4-diethylamino- | 0.0003 |
| phenyl)vinyl]-4-(4-isobutyl- | |
| phenyl)-3-methyl- | |
| thiazolium iodide | |
| Formula IV | 0.0002 |
| 2,3-dimethyl-4(p- | 0.02 |
| cyclohexylphenyl) | |
| thiazolium iodide | |
| 4-(4-isobutylphenyl)-3- | 0.0004 |
| methyl-2-[2-(4-pyrrolidin-1- | |
| yl-phenyl)vinyl]- | |
| thiazolium iodide | |

In the specification, there has been disclosed embodiments of the invention and, although specific terms are employed, they are used in a generic and descriptive sense only and not for purposes of limitation of the scope of the invention being set forth in the following claims.

What is claimed is:

1. A method of controlling fungi comprising administering a composition comprising: 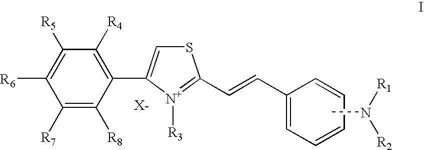
or a solvate thereof and
wherein the NR₁R₂ moiety is in the ortho, meta or para positions;
wherein X⁻ is an anion;
wherein R₁ and R₂ are independently selected from the group consisting of C₁₋₁₀ alkyl (linear or branched), alkenes (linear or branched), or wherein R₁ and R₂ taken together with the nitrogen atom to which they are attached form pyrrolidino or piperidino rings;
wherein R₃ is selected from the group consisting of C₁₋₁₀ alkyl (linear or branched), alkenes (linear or branched), alkynes, n-propyl, i-propyl, n-butyl, i-butyl, a polyalkylene glycol moiety, substituted and unsubstituted aryl moieties and substituted and unsubstituted benzyl moieties; and
wherein R₄ to R₈ are selected from the group consisting of hydrogen, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties.

2. The method according to claim 1, wherein said method of controlling fungi further comprises binding and containing the fungi in the same area.

3. The method according to claim 1, wherein said composition is administered before fungal growth occurs.

4. The method according to claim 1, wherein R₃ further comprises a metal and has the formula (CH₂)ₙ-MR₉, wherein n is a number from 1 to 6, M is an organometallic compound selected from the group consisting of tin, silicon, and germanium, and wherein R₉ is a selected from the group consisting of propyl, butyl, and alkyl, substituted or unsubstituted.

5. The method according to claim 1, wherein said composition is administered after fungal growth occurs.

6. The method according to claim 1, wherein said method further comprises administering organotin, organosilicon, or organogermanium.

7. The method according to claim 1, wherein R₃ further comprises an ultraviolet blocker, ultraviolet absorber or surfactant.

8. A method for treating agricultural fungal and/or bacterial infections comprising administering an effective amount a composition comprising: 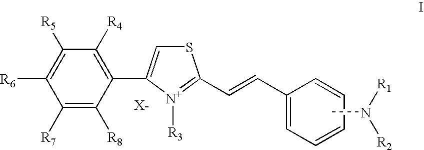
or a solvate thereof and
wherein the NR₁R₂ moiety is in the ortho, meta or para positions;
wherein X⁻ is an anion;
wherein R₁ and R₂ are independently selected from the group consisting of C₁₋₁₀ alkyl (linear or branched), alkenes (linear or branched), or wherein R₁ and R₂ taken together with the nitrogen atom to which they are attached form pyrrolidino or piperidino rings;
wherein R₃ is selected from the group consisting of C₁₋₁₀ alkyl (linear or branched), alkenes (linear or branched), alkynes, n-propyl, i-propyl, n-butyl, i-butyl, a polyalkylene glycol moiety, substituted and unsubstituted aryl moieties, substituted and unsubstituted benzyl moieties and (CH₂)ₙ-MR₉, wherein n is a number from 1 to 6, M is an organometallic compound selected from the group consisting of tin, silicon, and germanium, and wherein R₉ is selected from the group consisting of propyl, butyl and alkyl, substituted or unsubstituted; and
wherein R₄ to R₈ are selected from the group consisting of hydrogen, methyl, ethyl, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties.

9. The method according to claim 8, further comprising administering a fungicide and/or bacteriocide.

10. The method according to claim 8, further comprising administering an insecticide.

11. The method according to claim 8, wherein said composition is administered before fungal growth occurs.

12. The method according to claim 8, wherein said composition is administered after fungal growth occurs.

13. The method according to claim 8, further comprising administering organotin, organosilicon, or organogermanium.

14. The method according to claim 8, wherein said method of controlling fungi and/or bacteria further comprises binding and containing the fungi and/or bacteria in the same area.

15. The method according to claim 8, wherein said treating step is performed on a seed.

16. The method according to claim 8, wherein said treating step is performed on a plant.

17. The method according to claim 8, wherein said treating step is performed on a field used for growing crops.

18. A method of protecting a plant from fungal infection comprising contacting a plant during a stage of the growth of said plant with a compound comprising: 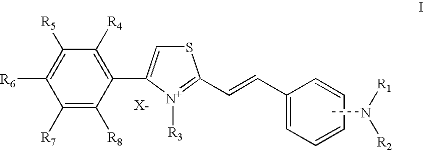
or a solvate thereof and
wherein the NR₁R₂ moiety is in the ortho, meta or para positions;
wherein X⁻ is an anion;
wherein R₁ and R₂ are independently selected from the group consisting of C₁₋₁₀ alkyl (linear or branched), alkenes (linear or branched), or wherein R₁ and R₂ taken together with the nitrogen atom to which they are attached form pyrrolidino or piperidino rings;
wherein R₃ is selected from the group consisting of C₁₋₁₀ alkyl (linear or branched), alkenes (linear or branched), alkynes, n-propyl, i-propyl, n-butyl, i-butyl, a polyalkylene glycol moiety, substituted and unsubstituted aryl moieties, substituted and unsubstituted benzyl moieties and (CH₂)ₙ-MR₉, wherein n is a number from 1 to 6, M is an organometallic compound selected from the group consisting of tin, silicon, and germanium, and wherein R₉ is selected from the group consisting of propyl, butyl and alkyl, substituted or unsubstituted; and
wherein R₄ to R₈ are selected from the group consisting of hydrogen, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties.

19. The method according to claim 18, further comprising administering a fungicide and/or bacteriocide.

20. The method according to claim 18, further comprising administering an insecticide.

21. The method according to claim 18, wherein a seed of said plant is immersed into a composition comprising said compound before said seed is planted in a growth medium for said plant.

22. The method according to claim 18, wherein said plant comprises plant seedlings or seeds and said plant is planted in a growth medium containing said compound.

23. A compound comprising Formula II 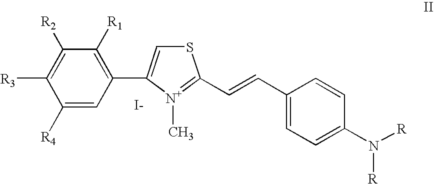
wherein R is a lower alkyl;
wherein R₁ is selected from the group consisting of hydrogen and a lower alkyl;
wherein R₂ is selected from the group consisting of hydrogen and a lower alkyl;
wherein R₃ is selected from the group consisting of hydrogen, alkoxy and a lower alkyl; and
wherein R₄ is selected from the group consisting of hydrogen and a lower alkyl;
or a solvate thereof.

24. A compound comprising: 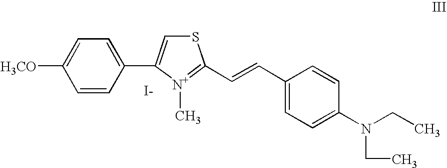
or a solvate thereof.

25. The compound of claim 24, wherein said compound is administered to treat a fungal or bacterial infection.

26. A compound comprising: 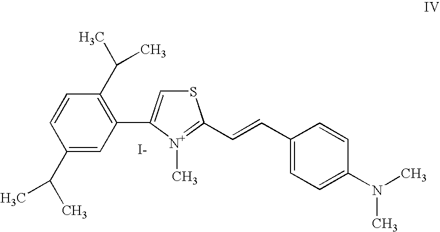
or a solvate thereof.

27. The compound claim 26, wherein said compound is administered to treat a fungal or bacterial infection.

28. A compound comprising: 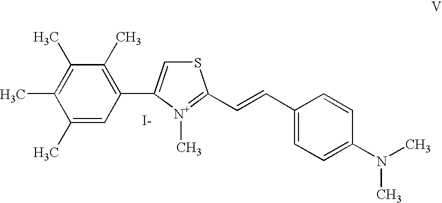
or a solvate thereof.

29. The compound of claim 28, wherein said compound is administered to treat a fungal or bacterial infection.

30. A method of controlling fungi and/or bacterial infections comprising administering a composition comprising: 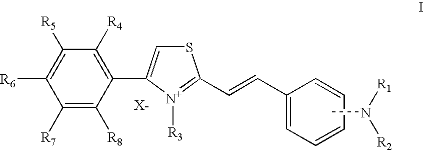
or a solvate thereof and
wherein the NR₁R₂ moiety is in the ortho, meta or para positions;
wherein X⁻ is an anion;
wherein R₁ and R₂ are independently selected from the group consisting of C₁₋₁₀ alkyl (linear or branched), alkenes (linear or branched), or wherein R₁ and R₂ taken together with the nitrogen atom to which they are attached form pyrrolidino or piperidino rings;
wherein R₃ is selected from the group consisting of C₁₋₁₀ alkyl (linear or branched), alkenes (linear or branched), alkynes, n-propyl, i-propyl, n-butyl, i-butyl, a polyalkylene glycol moiety, substituted and unsubstituted aryl moieties, substituted and unsubstituted benzyl moieties and (CH₂)ₙ-MR₉, wherein n is a number from 1 to 6, M is an organometallic compound selected from the group consisting of tin, silicon, and germanium, and wherein R₉ is selected from the group consisting of propyl, butyl and alkyl, substituted or unsubstituted; and
wherein R₄ to R₈ are selected from the group consisting of hydrogen, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties.

31. The method according to claim 30, wherein said method of controlling fungi and/or bacteria further comprises binding and containing the fungi and/or bacteria in the same area.

32. The method according to claim 30, wherein said composition is administered before fungal growth occurs.

33. The method according to claim 30, wherein R₃ is (CH₂)ₙ-MR₆, wherein n is a number from 1 to 6, M is an organometallic compound selected from the group consisting of tin, silicon, and germanium, and wherein R₆ is a selected from the group consisting of propyl, butyl, and alkyl, substituted or unsubstituted.

34. The method according to claim 30, wherein said composition is administered after fungal growth occurs.

35. The method according to claim 30, wherein said composition is administered to a substrate.

36. The method according to claim 35, wherein said substrate is selected from the group consisting of wood, air ducts, lumber, floorings, decks, buoys, seawalls, retaining walls, docks, pilings, watercrafts, boats, pipes, stucco, tiles, paint, insulation, roofs, roofing materials, building materials, metal, concrete and cement-based materials, plasters, asphalts, ceramics, stucco, sheetrock, grout, caulking, mortar, plastics, foam, glass, carpets, wallpaper, cloth, computer parts, food packaging, paper products, medical devices, petroleum processing, oil and natural gas extraction, metal working fluids, fasteners, adhesives, sealants, swimming pools, saunas, hot tubs, whirlpools, jacuzzis and spas, and surfaces thereof, wall coverings, siding materials, flooring, filtration systems and cooling towers.

37. The method according to claim 30, wherein said method comprises controlling fungi and/or bacteria by reducing fungal and/or bacterial growth in food packaging systems.

38. The method according to claim 30, wherein said method comprises controlling fungi and/or bacteria in medical products.

39. The method according to claim 30, wherein said method comprises controlling fungi and/or bacteria on a substrate.

40. The method according to claim 37, wherein said food packaging systems are selected from the group consisting of plastic, paper and foam.

41. The method according to claim 30, wherein said method further comprises administering organotin, organosilicon, or organogermanium.

42. The method according to claim 30, wherein R₃ further comprises an ultraviolet blocker or an ultraviolet absorber.

43. The method according to claim 42, wherein said ultraviolet blocker or an ultraviolet absorber is selected from the group consisting of

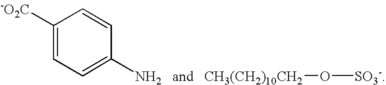

44. A microcapsule comprising a composition comprising formula (I) 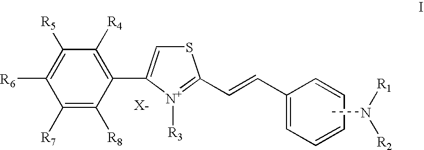
or a solvate thereof and
wherein the NR₁R₂ moiety is in the ortho, meta or para positions;
wherein X⁻ is an anion;
wherein R₁ and R₂ are independently selected from the group consisting of C₁₋₁₀ alkyl (linear or branched), alkenes (linear or branched), or wherein R₁ and R₂ taken together with the nitrogen atom to which they are attached form pyrrolidino or piperidino rings;
wherein R₃ is selected from the group consisting of C₁₋₁₀ alkyl (linear or branched), alkenes (linear or branched), alkynes, n-propyl, i-propyl, n-butyl, i-butyl, a polyalkylene glycol moiety, substituted and unsubstituted aryl moieties, substituted and unsubstituted benzyl moieties and (CH₂)ₙ-MR₉, wherein n is a number from 1 to 6, M is an organometallic compound selected from the group consisting of tin, silicon, and germanium, and wherein R₉ is selected from the group consisting of propyl, butyl and alkyl, substituted or unsubstituted; and
wherein R₄ to R₈ are selected from the group consisting of hydrogen, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties;
an excipient; and
a photosensitive material.

45. The microcapsule of claim 44, wherein the photosensitive material absorbs ultraviolet radiation.

46. The microcapsule of claim 44, wherein the photosensitive material blocks ultraviolet radiation.

47. The microcapsule of claim 44, wherein a ratio of the photosensitive material to the formula is 1:10.

48. A method for treating an inflammation in a subject comprising:
topically administering a composition comprising: 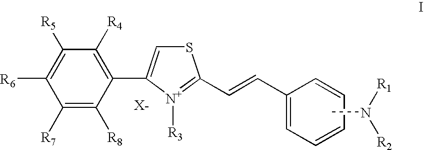
or a solvate thereof and
wherein the NR₁R₂ moiety is in the ortho, meta or para positions;
wherein X⁻ is an anion;
wherein R₁ and R₂ are independently selected from the group consisting of C₁₋₁₀ alkyl (linear or branched), alkenes (linear or branched), or wherein R₁ and R₂ taken together with the nitrogen atom to which they are attached form pyrrolidino or piperidino rings;
wherein R₃ is selected from the group consisting of C₁₋₁₀ alkyl (linear or branched), alkenes (linear or branched), alkynes, n-propyl, i-propyl, n-butyl, i-butyl, a polyalkylene glycol moiety, substituted and unsubstituted aryl moieties, substituted and unsubstituted benzyl moieties and (CH₂)ₙ-MR₉, wherein n is a number from 1 to 6, M is an organometallic compound selected from the group consisting of tin, silicon, and germanium, and wherein R₉ is selected from the group consisting of propyl, butyl and alkyl, substituted or unsubstituted; and
wherein R₄ to R₉ are selected from the group consisting of hydrogen, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties.

49. The method according to claim 48, wherein said inflammation is selected from the group consisting of allergic rhinitis, otitis, sinusitis, asthma, adult respiratory distress syndrome, bronchitis, laryngitis, thrush and cystic fibrosis.

50. The method according to claim 48, wherein said composition is administered to the skin or mucous membranes of the subject.

51. The method according to claim 48, wherein R₃ further comprises an ultraviolet blocker or an ultraviolet absorber.

52. The method according to claim 48, further comprising treating a fungal infection.

53. The method according to claim 52, wherein said fungal infection is selected form the group consisting of tinea pedis, tinea capitis, tinea corporis, tinea versicolor, nail fungal diseases, scalp disorders, tinea cruris, candidiasis, rhinosinusitis and allergic rhinitis.

54. A method for treating an immune disease in a subject comprising:
administering a composition comprising formula (I) 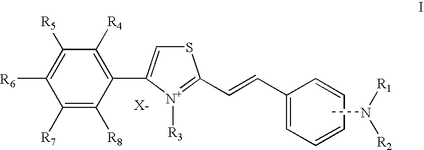
or a solvate thereof and
wherein the NR₁R₂ moiety is in the ortho, meta or para positions;
wherein X⁻ is an anion;
wherein R₁ and R₂ are independently selected from the group consisting of C₁₋₁₀ alkyl (linear or branched), alkenes (linear or branched), or wherein R₁ and R₂ taken together with the nitrogen atom to which they are attached form pyrrolidino or piperidino rings;
wherein R₃ is selected from the group consisting of C₁₋₁₀ alkyl (linear or branched), alkenes (linear or branched), alkynes, n-propyl, i-propyl, n-butyl, i-butyl, a polyalkylene glycol moiety, substituted and unsubstituted aryl moieties, substituted and unsubstituted benzyl moieties and (CH₂)ₙ-MR₉, wherein n is a number from 1 to 6, M is an organometallic compound selected from the group consisting of tin, silicon, and germanium, and wherein R₉ is selected from the group consisting of propyl, butyl and alkyl, substituted or unsubstituted; and
wherein R₄ to R₈ are selected from the group consisting of hydrogen, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties;
to a subject in need thereof.

55. The method according to claim 54, wherein said immune disease is selected from the group consisting of allergic rhinitis, otitis extema, sinusitis, asthma, adult respiratory distress syndrome, bronchitis, laryngitis, thrush, cystic fibrosis, rheumatoid arthritis, rheumatoid spondylitis, osteoarthritis, gouty arthritis, uveitis, conjunctivitis, inflammatory bowel conditions, Crohn's disease, ulcerative colitis, distal proctitis, psoriasis, eczema, dermatitis, allergic prurigo, topical fungal infections, gingivitis, periodontitis, coronary infarct damage, chronic inflammation, asthma, adult respiratory distress syndrome, rhinitis, chronic rhinosinusitis, orophoryngeal candidiasis, bronchitis, laryngitis, cystic fibrosis and smooth muscle proliferation disorders.

56. An article of manufacture comprising:
(a) a substrate; and
(b) a compound of formula (I) 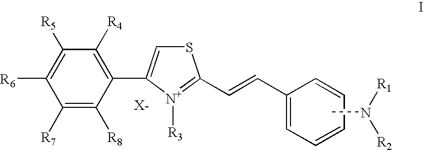
or a solvate thereof and
wherein the NR₁R₂ moiety is in the ortho, meta or para positions;
wherein X⁻ is an anion;
wherein R₁ and R₂ are independently selected from the group consisting of C₁₋₁₀ alkyl (linear or branched), alkenes (linear or branched), or wherein R₁ and R₂ taken together with the nitrogen atom to which they are attached form pyrrolidino or piperidino rings;
wherein R₃ is selected from the group consisting of C₁₋₁₀ alkyl (linear or branched), alkenes (linear or branched), alkynes, n-propyl, i-propyl, n-butyl, i-butyl, a polyalkylene glycol moiety, substituted and unsubstituted aryl moieties, substituted and unsubstituted benzyl moieties and (CH₂)ₙ-MR₉, wherein n is a number from 1 to 6, M is an organometallic compound selected from the group consisting of tin, silicon, and germanium, and wherein R₉ is selected from the group consisting of propyl, butyl and alkyl, substituted or unsubstituted; and
wherein R₄ to R₈ are selected from the group consisting of hydrogen, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties.

57. The article of manufacture according to claim 56, wherein the substrate is used in the manufacture of an article selected from the group consisting of wood, air ducts, lumber, floorings, decks, buoys, seawalls, retaining walls, docks, pilings, watercrafts, boats, pipes, stucco, tiles, paint, insulation, roofs, roofing materials, building materials, metal, concrete and cement-based materials, plasters, asphalts, ceramics, stucco, sheetrock, grout, caulking, mortar, plastics, foam, glass, carpets, wallpaper, cloth, computer parts, food packaging, paper products, medical devices, petroleum processing, oil and natural gas extraction, metal working fluids, fasteners, adhesives, sealants, swimming pools, saunas, hot tubs, whirlpools, jacuzzis and spas, and surfaces thereof, wall coverings, siding materials, flooring, filtration systems, cooling towers, personal care and/or hygiene products and cosmetics.

58. A method of controlling algal, fungal, bacterial, viral, and/or parasitic growth on a substrate, said method comprising applying a composition to the substrate in an amount effective to control the growth of algae, fungi, bacteria, viruses, and/or parasites wherein said composition comprises:
(a) a compound of formula I 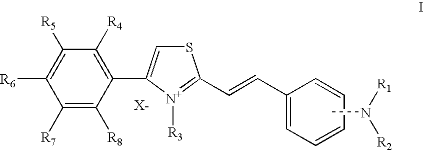
or a solvate thereof and
wherein the NR₁R₂ moiety is in the ortho, meta or para positions;
wherein X⁻ is an anion;
wherein R₁ and R₂ are independently selected from the group consisting of C₁₋₁₀ alkyl (linear or branched), alkenes (linear or branched), or wherein R₁ and R₂ taken together with the nitrogen atom to which they are attached form pyrrolidino or piperidino rings;
wherein R₃ is selected from the group consisting of C₁₋₁₀ alkyl (linear or branched), alkenes (linear or branched), alkynes, n-propyl, i-propyl, n-butyl, i-butyl, a polyalkylene glycol moiety, substituted and unsubstituted aryl moieties, substituted and unsubstituted benzyl moieties and (CH₂)ₙ-MR₉, wherein n is a number from 1 to 6, M is an organometallic compound selected from the group consisting of tin, silicon, and germanium, and wherein R₉ is selected from the group consisting of propyl, butyl and alkyl, substituted or unsubstituted, and wherein R₄ to R₈ are selected from the group consisting of hydrogen, C₁₋₁₀ alkyl (linear or branched), alkenes (linear or branched), alkynes, n-propyl, i-propyl, n-butyl, i-butyl, substituted and unsubstituted aryl moieties and substituted and unsubstituted benzyl moieties; and
(b) a cosmetically, agriculturally or industrially acceptable carrier, excipient or diluent.
